(19) United States
(12) Patent Application Publication
(10) Pub. No.: US 2026/0241034 A1
(43) Pub. Date: Aug. 20, 2026

(54) MULTI-TERMINUS POLYA POLYNUCLEOTIDES

(71) Applicant: Convergence Bio, Inc., Cambridge, MA (US)

(72) Inventors: Muthiah MANOHARAN, Cambridge, MA (US); Chong WANG, Cambridge, MA (US); Anilkumar KORE, Cambridge, MA (US)

(21) Appl. No.: 19/637,271

(22) Filed: Apr. 2, 2026

Related U.S. Application Data
(63) Continuation of application No. PCT/US2024/049595, filed on Oct. 2, 2024.
(60) Provisional application No. 63/587,335, filed on Oct. 2, 2023.

(51) Int. Cl. A61K 48/00; C07H 21/02

(52) U.S. Cl. CPC A61K 48/005; C07H 21/02

(57) ABSTRACT

Provided herein are modified polynucleotides useful for increasing the stability of an mRNA molecule and/or production of a therapeutic protein.

### CROSS-REFERENCE

This application is a continuation of International Patent Application No. PCT/US2024/049595, filed on Oct. 2, 2024, which claims the benefit of priority to U.S. Provisional Application No. 63/587,335 filed on Oct. 2, 2023, each of which is herein incorporated by reference.

### BACKGROUND

By relying on the patient's body to make the desired protein, mRNA drugs provide, in theory, an approach for robust and tunable production of a therapeutic protein. However, the current application of mRNA drugs is limited, especially when compared to its potential to serve as a platform for an effective, scalable, and manufacture-friendly therapeutic modality. Notably, development and use of mRNA drugs, while promising, is not without challenges and hurdles that prevent unlocking the fuller potential of mRNA-based drugs as a robust and effective therapeutic modality.

### SUMMARY

mRNA molecules are generally unstable and can be quickly degraded by enzymes in the body. This instability poses a significant hurdle for mRNA drugs. This hurdle also impacts the duration of therapeutic action (e.g., production of a therapeutic protein), wherein the current duration of therapeutic protein translation is too short, thereby, in certain instances, limiting efficacy.

Provided herein are modified polynucleotides useful for increasing the stability of an mRNA molecule and/or production of a therapeutic protein. In some embodiments, provided herein are multi-terminus polynucleotides comprising a first polyA polynucleotide linked to a second polyA polynucleotide, wherein the multi-terminus polyA polynucleotide comprises a formula represented by the structure:

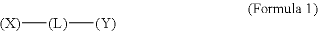

wherein:
   (X) is a first functionalized nucleotide within the first polyA polynucleotide, wherein the functionalized nucleotide comprises an amino modification, an alkoxyamino modification, a thiol modification, a carboxyl modification, or an aldehyde modification;
   (L) is a linker;
   (Y) is a second functionalized nucleotide within the second polyA polynucleotide, wherein the functionalized nucleotide comprises an amino modification, an alkoxyamino modification, a carboxyl modification, or an aldehyde modification;
   the first functionalized nucleotide is linked to the linker through a linkage comprising an amide linkage, an oxime linkage, or a thioether linkage, and
   the linker is linked to the second functionalized nucleotide through a linkage comprising an amide linkage, an oxime linkage, or a thioether linkage.

In some embodiments, the multi-terminus polyA polynucleotide comprises a formula represented by the structure:

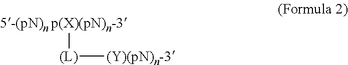

wherein:
   (X) is a first functionalized nucleotide within the first polyA polynucleotide, wherein the functionalized nucleotide comprises an amino modification, an alkoxyamino modification, a thiol modification, a carboxyl modification, or an aldehyde modification;
   (L) is a linker;
   (Y) is a second functionalized nucleotide at the 5′ position of a second polyA polynucleotide, wherein the functionalized nucleotide comprises an amino modification, an alkoxyamino modification, a carboxyl modification, or an aldehyde modification;
   each (p) is independently an inter-nucleoside linkage (e.g., phosphodiester or phosphorothioate bond) or terminal phosphate or phosphorothioate;
   each (N) is independently a nucleotide or a modified nucleotide;
   each (n) is independently an integer equal to or greater than 1;
   first functionalized nucleotide is linked to the linker through a linkage comprising an amide linkage, an oxime linkage, a thioether linkage, and
   the linker is linked to the second functionalized nucleotide through a linkage comprising an amide linkage, a oxime linkage, a thioether linkage.
      In such embodiments, the polarity of the second polyA polynucleotide is 5′→3′ (proximal to distal) relative to its linkages to (X) through (L).

In some embodiments, the multi-terminus polyA polynucleotide comprises a formula represented by the structure:

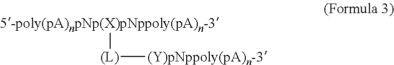

wherein:
   (X) is a first functionalized nucleotide within a first polyA polynucleotide, wherein the functionalized nucleotide comprises an amino modification, an alkoxyamino modification, a thiol modification, a carboxyl modification, or an aldehyde modification;
   (L) is a linker;
   (Y) is a second functionalized nucleotide at the 5′ position of a second polyA polynucleotide, wherein the functionalized nucleotide comprises an amino modification, an alkoxyamino modification, a carboxyl modification, or an aldehyde modification;
   each (p) is independently an inter-nucleoside linkage (e.g., phosphodiester or phosphorothioate bond) or terminal phosphate or phosphorothioate;
   each (N) is independently a nucleotide or a modified nucleotide;
   each (A) is independently an adenine nucleotide or a modified adenine nucleotide;
   each (n) is independently an integer equal to or greater than 1;
   the first functionalized nucleotide is linked to the linker through a linkage comprising an amide linkage, an oxime linkage, a thioether linkage, and
   the linker is linked to the functionalized nucleotide is linked to the linker through a linkage comprising an amide linkage, a oxime linkage, a thioether linkage.

In some embodiments, provided are compositions of multi-terminus polynucleotides (e.g., multi-terminus mRNAs) comprising a first polyA polynucleotide linked to a second polyA polynucleotide, wherein the composition is substantially free of copper). In certain embodiments, the multi-terminus polynucleotides comprise a formula represented by the structures in any one of Formulas 1-8. In such embodiments, the first and the second functionalized nucleotide comprise an amino modification, an alkoxyamino modification, a thiol modification, a carboxyl modification, an aldehyde modification, an azide modification, or an alkyne modification. Also in such embodiments, first functionalized nucleotide is linked to the linker through a linkage comprising an amide linkage, an oxime linkage, a triazole linkage, or a thioether linkage, and second functionalized nucleotide is linked to the linker through a linkage comprising an amide linkage, an oxime linkage, a triazole linkage, or a thioether linkage.

In some embodiments, provided are methods of increasing translation of a polypeptide, the method comprising: contacting a cell with (i) a messenger ribonucleic acid molecule comprising the multi-terminus polyA polynucleotides described herein or (ii) the modified ribonucleic acid molecules described herein.

In some embodiments, increasing translation comprises increasing the total amount of the polypeptide. In some embodiments, increasing translation comprises increasing the duration of protein translation.

### BRIEF DESCRIPTION OF THE DRAWINGS

The novel features of the disclosure are set forth with particularity in the detailed description and appended claims. A better understanding of the features and advantages of the present invention will be obtained by reference to the following detailed description that sets forth illustrative embodiments, in which the principles of the invention are utilized, and the accompanying drawings of which:

FIG. 1 shows an exemplary modified mRNA construct having a multi-terminus polynucleotide modifications.

FIG. 2 shows an exemplary modified mRNA construct having a multi-terminus polynucleotide modifications.

FIG. 3A shows an exemplary 5′-amine MOE-PS modified oligonucleotide.

FIG. 3B shows an exemplary reaction schematic for preparing activated NHS ester modified oligonucleotide.

FIG. 3C shows an exemplary reaction schematic.

FIGS. 4A and 4B show exemplary LC/Q-TOF analysis and mass determination of mono branched MOE-PS oligo. LC/Q-TOF analysis confirmed the mass of mono branched MOE-PS oligo.

FIG. 5 shows exemplary data demonstrating mRNAs having branched polyA tails using amide chemistries improves mRNA stability and increased protein expression

FIG. 6 shows an exemplary general schematic of oligo synthesis.

FIG. 7A shows an exemplary reaction schematic of deprotection of 5′-amine oligonucleotides prior conjugation.

FIG. 7B shows an exemplary reaction schematic of bioconjugation of 5′-Azide via Amine/NHS ester.

FIG. 7C shows an exemplary reaction schematic for conjugation of DBCO-NHS ester with internal amino RNA.

FIG. 7D shows an exemplary conjugation of DBCO-modified oligo RNA with azide-modified RNA branches.

FIG. 8 shows an exemplary Gel-based RNase H assay indicates conjugation occurring on RNA.

FIG. 9 shows exemplary data demonstrating mRNAs having branched polyA tails using copper-free chemistries improves mRNA stability and increased protein expression.

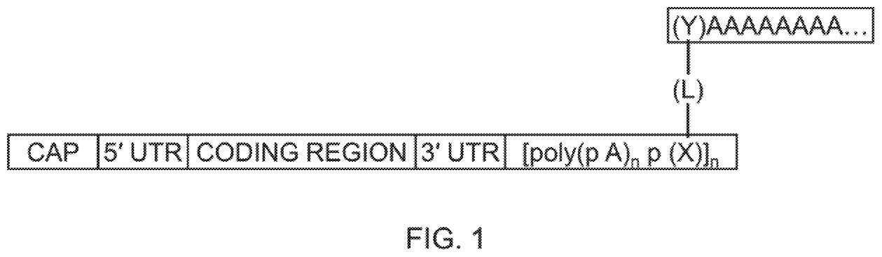

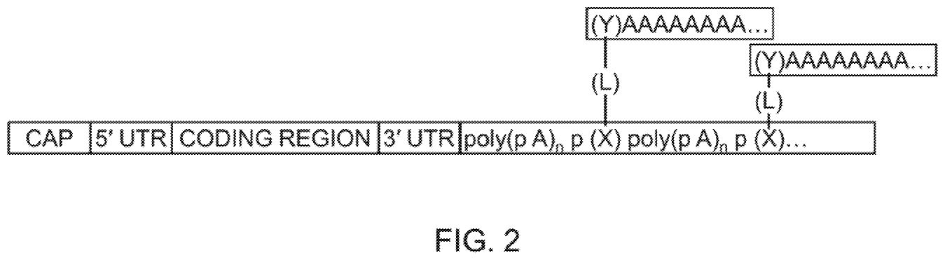

### DETAILED DESCRIPTION

### Multi-Terminus Polynucleotides

In some embodiments, provided herein are multi-terminus polynucleotides comprising a first polyA polynucleotide linked to a second polyA polynucleotide, wherein the multi-terminus polyA polynucleotide comprises a formula represented by the structure:

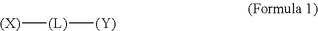

wherein:
   (X) is a first functionalized nucleotide within the first polyA polynucleotide, wherein the functionalized nucleotide comprises an amino modification, an alkoxyamino modification, a thiol modification, a carboxyl modification, or an aldehyde modification;
   (L) is a linker;
   (Y) is a second functionalized nucleotide within the second polyA polynucleotide, wherein the functionalized nucleotide comprises an amino modification, an alkoxyamino modification, a carboxyl modification, or an aldehyde modification;
   the first functionalized nucleotide is linked to the linker through a linkage comprising an amide linkage, an oxime linkage, or a thioether linkage, and
   the linker is linked to the second functionalized nucleotide through a linkage comprising an amide linkage, an oxime linkage, or a thioether linkage.

In some embodiments, a multi-terminus polynucleotide generally refers to and encompasses a first polyA polynucleotide linked to a second polyA polynucleotide. In certain embodiments the multi-terminus polynucleotide comprises a first polyA polynucleotide linked to 1 or more independent polyA polynucleotides. In certain embodiments the multi-terminus polynucleotide comprises a first polyA polynucleotide linked to 2 or more independent polyA polynucleotides. In certain embodiments the multi-terminus polynucleotide comprises a first polyA polynucleotide linked to 3 or more independent polyA polynucleotides. In certain embodiments the multi-terminus polynucleotide comprises a first polyA polynucleotide linked to 4 or more independent polyA polynucleotides. In certain embodiments the multi-terminus polynucleotide comprises a first polyA polynucleotide linked to 5 or more independent polyA polynucleotides.

In some embodiments, the polyA polynucleotide comprises a polynucleotide having a nucleobase sequence, wherein the nucleobase sequence comprises 75% or greater adenine (A) nucleobases (e.g., including naturally occurring adenine nucleotides, modified adenine nucleotides, and/or modified adenine bases). In certain embodiments, the nucleobase sequence comprises 80% or greater adenine (A) nucleobases. In certain embodiments, the nucleobase sequence comprises 90% or greater adenine (A) nucleobases. In certain embodiments, the nucleobase sequence comprises 95% or greater adenine (A) nucleobases. In some embodiments, polyA generally refers to and encompasses polymer a formula represented by the structure: (pA)ₙ wherein (pA) is adenine monophosphate or a modified adenine monophosphate, and (n) is an integer great than 1 (e.g., 2, 5, 10, 25, 50, 100, etc.). In some embodiments, polyA polynucleotide generally refers to and encompasses a polyA tail. In certain embodiments, polyA tail generally refers to and encompasses a chain of adenine nucleotides (e.g., 75% or greater adenine) that is added to a messenger ribonucleic acid (mRNA) molecule during ribonucleic acid (RNA).

In some embodiments, the polyA polynucleotide comprises a modified nucleotide (e.g., base modification, sugar modification, and/or inter-nucleotide linkage modification). In some embodiments, the polyA polynucleotide comprises one or more modified nucleotides. In certain embodiments, a modified nucleotide comprises a nucleotide comprising a modification, e.g., a non-naturally occurring nucleotide. In certain embodiments, the modification is a base modification. In certain embodiments, the modification is a sugar modification. In certain embodiments, the modification is an inter-nucleotide linkage modification.

In some embodiments, (X) is located at a non-terminal position of the first polyA polynucleotide. In some embodiments, (Y) is located at a terminal or a non-terminal position of the second polyA polynucleotide. In certain embodiments, (Y) is located at a terminal position. In certain embodiments, (Y) is located at a non-terminal position.

In some embodiments, the multi-terminus polyA polynucleotide comprises a formula represented by the structure:

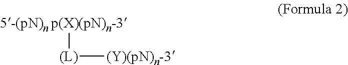

wherein:
   (X) is a first functionalized nucleotide within the first polyA polynucleotide, wherein the functionalized nucleotide comprises an amino modification, an alkoxyamino modification, a thiol modification, a carboxyl modification, or an aldehyde modification;
   (L) is a linker;
   (Y) is a second functionalized nucleotide at the 5′ position of a second polyA polynucleotide, wherein the functionalized nucleotide comprises an amino modification, an alkoxyamino modification, a carboxyl modification, or an aldehyde modification;
   each (p) is independently an inter-nucleoside linkage (e.g., phosphodiester or phosphorothioate bond) or terminal phosphate or phosphorothioate;
   each (N) is independently a nucleotide or a modified nucleotide;
   each (n) is independently an integer equal to or greater than 1;
   first functionalized nucleotide is linked to the linker through a linkage comprising an amide linkage, an oxime linkage, a thioether linkage, and
   the linker is linked to the second functionalized nucleotide through a linkage comprising an amide linkage, a oxime linkage, a thioether linkage.
      In such embodiments, the polarity of the second polyA polynucleotide is 5′→3′ (proximal to distal) relative to its linkages to (X) through (L).

In some embodiments, the multi-terminus polyA polynucleotide comprises a formula represented by the structure:

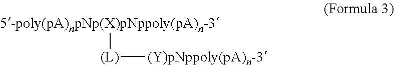

wherein:
   (X) is a first functionalized nucleotide within a first polyA polynucleotide, wherein the functionalized nucleotide comprises an amino modification, an alkoxyamino modification, a thiol modification, a carboxyl modification, or an aldehyde modification;
   (L) is a linker;
   (Y) is a second functionalized nucleotide at the 5′ position of a second polyA polynucleotide, wherein the functionalized nucleotide comprises an amino modification, an alkoxyamino modification, a carboxyl modification, or an aldehyde modification;
   each (p) is independently an inter-nucleoside linkage (e.g., phosphodiester or phosphorothioate bond) or terminal phosphate or phosphorothioate;
   each (N) is independently a nucleotide or a modified nucleotide;
   each (A) is independently an adenine nucleotide or a modified adenine nucleotide;
   each (n) is independently an integer equal to or greater than 1;
   the first functionalized nucleotide is linked to the linker through a linkage comprising an amide linkage, an oxime linkage, a thioether linkage, and
   the linker is linked to the functionalized nucleotide is linked to the linker through a linkage comprising an amide linkage, a oxime linkage, a thioether linkage.
      In such embodiments, the polarity of the second polyA polynucleotide is 5′→3′ (proximal to distal) relative to its linkages to (X) through (L).

In some embodiments, the multi-terminus polyA polynucleotide comprises a formula represented by the structure:

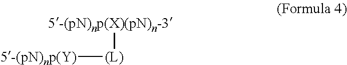

wherein:
   (X) is a first functionalized nucleotide within a first polyA polynucleotide (e.g., wherein the functionalized nucleotide comprises an amino modification, an alkoxyamino modification, a thiol modification, a carboxyl modification, or an aldehyde modification);
   (L) is a linker;
   (Y) is a second functionalized nucleotide at the 3′ position of a second polyA polynucleotide (e.g., wherein the functionalized nucleotide comprises an amino modification, an alkoxyamino modification, a thiol modification, a carboxyl modification, or an aldehyde modification);
   each (p) is independently an inter-nucleoside linkage (e.g., phosphodiester or phosphorothioate bond) or terminal phosphate or phosphorothioate;
   each (N) is independently a nucleotide or a modified nucleotide;
   each (n) is independently an integer equal to or greater than 1;
   the first functionalized nucleotide is linked to the linker (e.g., through a linkage comprising an amide linkage, an oxime linkage, a thioether linkage); and
   the linker is linked to the second functionalized nucleotide (e.g., through a linkage comprising an amide linkage, an oxime linkage, a thioether linkage).
      In such embodiments, the polarity of the second polyA polynucleotide is 3′→5′ (proximal to distal) relative to its linkages to (X) through (L).

In some embodiments, the multi-terminus polyA polynucleotide comprises a formula represented by the structure:

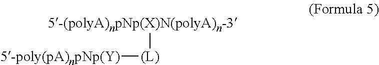

wherein:
   (X) is a first functionalized nucleotide within a first polyA polynucleotide (e.g., wherein the functionalized nucleotide comprises an amino modification, an alkoxyamino modification, a thiol modification, a carboxyl modification, or an aldehyde modification);
   (L) is a linker;
   (Y) is a second functionalized nucleotide at the 3′ position of a second polyA polynucleotide (e.g., wherein the functionalized nucleotide comprises an amino modification, an alkoxyamino modification, a thiol modification, a carboxyl modification, or an aldehyde modification);
   each (p) is independently an inter-nucleoside linkage (e.g., phosphodiester or phosphorothioate bond) or terminal phosphate or phosphorothioate;
   each (N) is independently a nucleotide or a modified nucleotide;
   each (A) is independently an adenine nucleotide or a modified adenine nucleotide;
   each (n) is independently an integer equal to or greater than 1;
   the first functionalized nucleotide is linked to the linker (e.g., through a linkage comprising an amide linkage, an oxime linkage, a thioether linkage), and
   the linker is linked to the second functionalized nucleotide is linked to the linker (e.g., through a linkage comprising an amide linkage, an oxime linkage, a thioether linkage).
      In such embodiments, the polarity of the second polyA polynucleotide is 3′→5′ (proximal to distal) relative to its linkages to (X) through (L).

In some embodiments, the multi-terminus polyA polynucleotide comprises a formula represented by the structure:

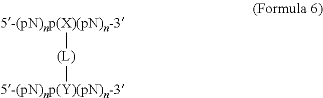

wherein:
   (X) is a first functionalized nucleotide within a first polyA polynucleotide, wherein the functionalized nucleotide comprises an amino modification, an alkoxyamino modification, a thiol modification, a carboxyl modification, or an aldehyde modification;
   (L) is a linker;
   (Y) is a second functionalized nucleotide within a second polyA polynucleotide, wherein the functionalized nucleotide comprises an amino modification, an alkoxyamino modification, a carboxyl modification, or an aldehyde modification;
   each (p) is independently an inter-nucleoside linkage (e.g., phosphodiester or phosphorothioate bond) or terminal phosphate or phosphorothioate;
   each (N) is independently a nucleotide or a modified nucleotide;
   each (n) is independently an integer equal to or greater than 1;
   the first functionalized nucleotide is linked to the linker through a linkage comprising an amide linkage, an oxime linkage, a thioether linkage, and
   the linker is linked to the second functionalized nucleotide through a linkage comprising an amide linkage, a oxime linkage, a thioether linkage.

In some embodiments, the multi-terminus polyA polynucleotide comprises a formula represented by the structure:

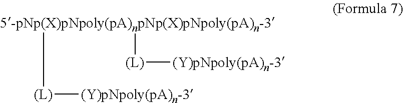

wherein:
   each (X) is independently a first functionalized nucleotide within a first polyA polynucleotide, wherein the functionalized nucleotide comprises an amino modification, an alkoxyamino modification, a thiol modification, a carboxyl modification, or an aldehyde modification;
   each (L) is independently a linker;
   each (Y) is independently a second functionalized nucleotide at the 5′ position of a second polyA polynucleotide, wherein the functionalized nucleotide comprises an amino modification, an alkoxyamino modification, a carboxyl modification, or an aldehyde modification;
   each (p) is independently an inter-nucleoside linkage (e.g., phosphodiester or phosphorothioate bond) or terminal phosphate or thiophosphate;
   each (N) is independently a nucleotide or a modified nucleotide;
   each (A) is independently an adenine nucleotide or a modified adenine nucleotide;
   each (n) is independently an integer equal to or greater than 1;
   the first functionalized nucleotide is linked to the linker through a linkage comprising an amide linkage, an oxime linkage, a thioether linkage, and
   the linker is linked to the second functionalized nucleotide through a linkage comprising an amide linkage, a oxime linkage, a thioether linkage.
      In such embodiments, the polarity of the second polyA polynucleotide is 5′→3′ (proximal to distal) relative to its linkages to (X) through (L).

In some embodiments, the multi-terminus polyA polynucleotide comprises a formula represented by the structure:

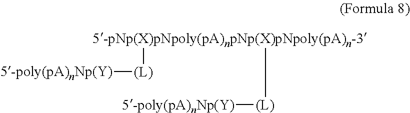

wherein:
   each (X) is independently a first functionalized nucleotide within a first polyA polynucleotide (e.g., wherein the functionalized nucleotide comprises an amino modification, an alkoxyamino modification, a thiol modification, a carboxyl modification, or an aldehyde modification);
   each (L) is independently a linker;
   each (Y) is independently a second functionalized nucleotide at the 3′ position of a second polyA polynucleotide (e.g., wherein the functionalized nucleotide comprises an amino modification, an alkoxyamino modification, a thiol modification, a carboxyl modification, or an aldehyde modification); each (p) is independently an inter-nucleoside linkage (e.g., phosphodiester or phosphorothioate bond) or terminal phosphate or phosphorothioate;
   each (N) is independently a nucleotide or a modified nucleotide;
   each (A) is independently an adenine nucleotide or a modified adenine nucleotide;
   each (n) is independently an integer equal to or greater than 1;
   the first functionalized nucleotide is linked to the linker (e.g., through a linkage comprising an amide linkage, an oxime linkage, a thioether linkage); and
   the linker is linked to the second functionalized nucleotide (e.g., through a linkage comprising an amide linkage, an oxime linkage, a thioether linkage).
      In such embodiments, the polarity of the second polyA polynucleotide is 3′→5′ (proximal to distal) relative to its linkages to (X) through (L).

In some embodiments, a functionalized nucleotide comprises a modification that permits the linkage (e.g., conjugation) to the linker, wherein the functionalized nucleotide comprises an amino modification, an alkoxyamino modification, a thiol modification, a carboxyl modification, or an aldehyde modification. In certain embodiments, the functional nucleotide comprises an amino modification (e.g., facilitating a linkage to the linker through an amide linkage). In certain embodiments, the functional nucleotide comprises a carboxyl modification (e.g., facilitating a linkage to the linker through an amide linkage). In certain embodiments, the functional nucleotide comprises an alkoxyamino modification (e.g., facilitating a linkage to the linker through an oxime linkage). In certain embodiments, the functional nucleotide comprises a thiol modification (e.g., facilitating a linkage to the linker through a thioether or disulfide linkage).

In certain embodiments, the first polyA polynucleotide comprises 2 or more functionalized nucleotides (e.g., 2 or more of (X)). In certain embodiments, the first polyA polynucleotide comprises 3 or more functionalized nucleotides. In certain embodiments, the first polyA polynucleotide comprises 4 or more functionalized nucleotides. In certain embodiments, the first polyA polynucleotide comprises 5 or more functionalized nucleotides.

In some embodiments, a polyA polynucleotide comprises about 10 to about 300 nucleotides. In some embodiments, a polyA polynucleotide comprises 10 or more nucleotides. In some embodiments, a polyA polynucleotide comprises 20 or more nucleotides. In some embodiments, a polyA polynucleotide comprises 30 or more nucleotides. In some embodiments, a polyA polynucleotide comprises 40 or more nucleotides. In some embodiments, a polyA polynucleotide comprises 50 or more nucleotides. In some embodiments, a polyA polynucleotide comprises 100 or more nucleotides. In some embodiments, a polyA polynucleotide comprises 150 or more nucleotides. In some embodiments, a polyA polynucleotide comprises 200 or more nucleotides. In some embodiments, a polyA polynucleotide comprises 250 or more nucleotides. In some embodiments, a polyA polynucleotide comprises 300 or more nucleotides.

### Functionalized Nucleotides

In some embodiments, a functionalized nucleotide comprises a functionalizing modification within the base or sugar-backbone. In certain embodiments, the functionalized nucleotide comprises an amino modification, an alkoxyamino modification, a thiol modification, a carboxyl modification, or an aldehyde modification. In certain embodiments, the functionalizing modification is within the nucleobase. In some embodiments, a functionalized nucleotide comprises a modification that permits the linkage (e.g., conjugation) to the linker, wherein the functionalized nucleotide comprises an amino modification, an alkoxyamino modification, a thiol modification, a carboxyl modification, or an aldehyde modification. In certain embodiments, the functional nucleotide comprises an amino modification (e.g., facilitating a linkage to the linker through an amide linkage). In certain embodiments, the functional nucleotide comprises a carboxyl modification (e.g., facilitating a linkage to the linker through an amide linkage). In certain embodiments, the functional nucleotide comprises an alkoxyamino modification (e.g., facilitating a linkage to the linker through an oxime linkage). In certain embodiments, the functional nucleotide comprises a thiol modification (e.g., facilitating a linkage to the linker through a thioether or disulfide linkage). For example, non-limiting functionalizing base modifications are shown in Table 1. In certain embodiments, the functionalizing modification comprises any one of the nucleobases selected from Table 1.

TABLE 1

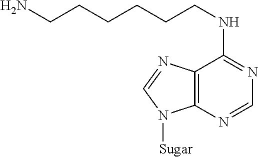

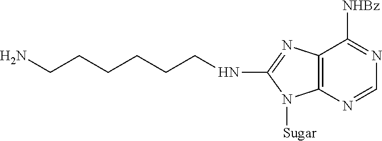

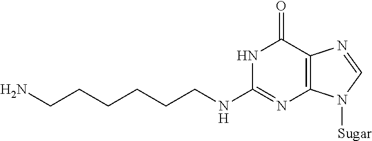

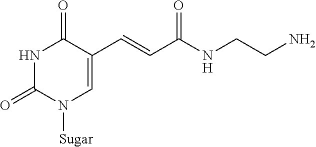

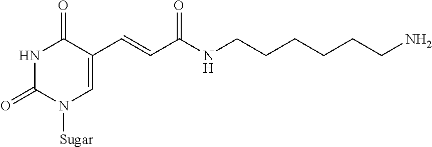

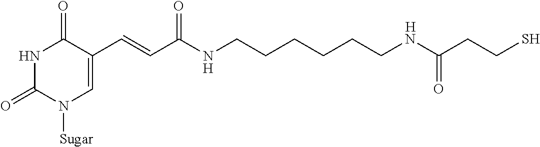

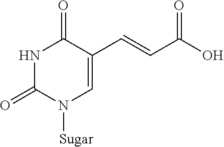

In certain embodiments, the functionalized nucleotide comprises a functionalizing modification within the ribose sugar. In certain embodiments, the modification is at the 2′ or 3′ position. For example, non-limiting ribose modifications for functionalized nucleotides are shown in Table 2. In certain embodiments, the functionalizing modification comprises any one of the ribose modifications selected from Table 2.

TABLE 2

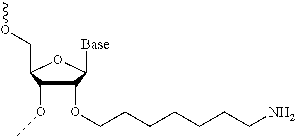

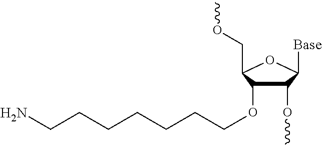

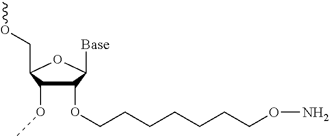

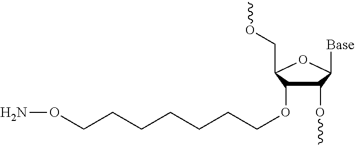

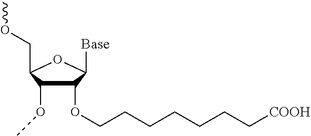

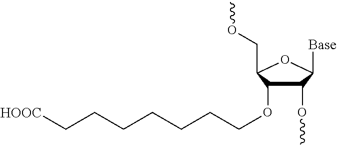

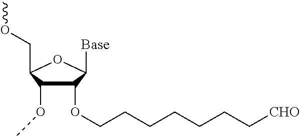

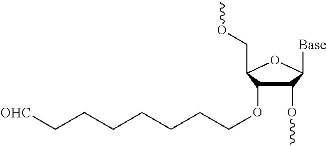

In certain instances, the functionalizing modifications of Table 1 and Table 2 are useful for functionalized nucleotides located at non-terminal positions (e.g., pN p (X) pN).

In certain embodiments, the functionalized nucleotide comprises a functionalizing modification within a nucleotide phosphate (e.g., 5′ or 3′ modifications). In certain instances, the nucleotide comprises a functionalizing modification of a 5′ phosphate. In certain instances, the nucleotide comprises a functionalizing modification of a 3′ phosphate. For example, non-limiting 5′ or 3′ modifications for functionalized nucleotides are shown in Table 3. In certain embodiments, the functionalizing modification comprises any one of the phosphate modifications selected from Table 3.

TABLE 3

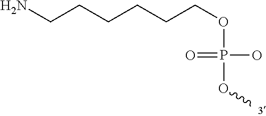

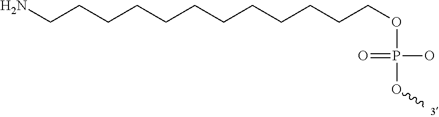

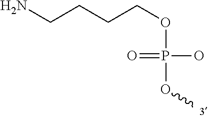

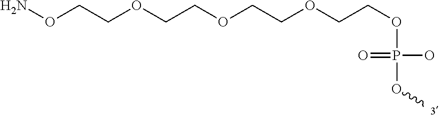

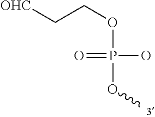

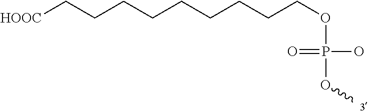

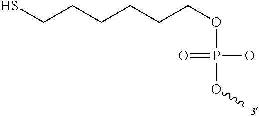

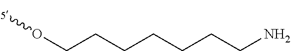

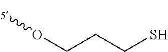

In some embodiments the functionalized nucleotide further comprises an additional nucleotide modification. In certain embodiments, the additional nucleotide modification is or comprises a sugar modification or inter-nucleotide linkage modification. In certain embodiments, the additional sugar modification comprises a 2′ OMe modification, a 2′ MOE modification, 2′ F modification, an LNA modification, a PMO modification, or an HNA modification. In certain embodiments, the additional inter-nucleotide linkage modification comprises a phosphorothioate modification, a boranothioate modification, a phNA modification, or a PNA modification.

### Modified Nucleotides

In some embodiments, the polyA polynucleotide further comprises one or more modified nucleotides (e.g., in addition to the functionalized nucleotides). In certain embodiments, the modified nucleotide comprises a nucleotide modification wherein the nucleotide modification a base modification, sugar modification, or inter-nucleotide linkage modification. In certain embodiments, the sugar modification comprises a 2′ OMe modification, a 2′ MOE modification, 2′ F modification, an LNA modification, a PMO modification, a GNA modification, or an HNA modification. In certain embodiments, the additional inter-nucleotide linkage modification comprises a phosphorothioate modification, a boranothioate modification, a phNA modification, or a PNA modification. In certain embodiments, the inter-nucleotide linkage modification comprises a phosphorothioate modification, a boranothioate modification, a phNA modification, or a PNA modification.

In some embodiments, the poly polynucleotide comprises a chain terminating modification at the 3′ end of the polyA polynucleotide. In certain embodiments, the chain terminating modification comprises a one or more sugar modifications (e.g., 2′ OMe modification, a 2′ MOE modification, 2′ F modification, an LNA modification, a PMO modification, or an HNA modification) of nucleotides adjacent to the 3′ terminus (e.g., within 20, 10, 5, or 2 positions of the 3′ terminus). In certain embodiments, the chain terminating modification comprises an inter-nucleotide linkage modification (e.g., a phosphorothioate modification, a boranothioate modification, a phNA modification, or a PNA modification) of nucleotides adjacent to the 3′ terminus (e.g., within 20, 10, 5, or 2 positions of the 3′ terminus). In certain embodiments, the chain terminating modification comprises a modification of secondary structure (e.g., a G4 quadraplex) at the 3′ end of the polyA. polynucleotide. In certain embodiments, the chain terminating modification comprises an inverted nucleotide (e.g., inverted thymidine) or a dideoxy nucleotide (e.g., ddC).

### Linkers

In some embodiments, a linker described herein (e.g., L) is a non-releasable linker, such as a linker that does not (e.g., readily) hydrolyze, such as in an aqueous or biological environment. In some embodiments, a linker described herein (e.g., L) is hydrolytically stable, such as in the presence of a buffer and/or (hydrolytic) proteins.

In some embodiments, a linker described herein (e.g., L) comprises one or more linker group, each linker group being independently selected from the group consisting of substituted or unsubstituted alkyl, substituted or unsubstituted heteroalkyl, substituted or unsubstituted carbocyclyl, substituted or unsubstituted heterocyclyl, substituted or unsubstituted aryl, and substituted or unsubstituted heteroaryl. In some embodiments, the linker (L) comprises one or more linker group, each linker group being independently selected from the group consisting of substituted or unsubstituted alkyl, substituted or unsubstituted heteroalkyl, substituted or unsubstituted heterocyclyl (e.g., a modified or unmodified ribose), and substituted or unsubstituted heteroaryl (e.g., a modified or unmodified adenine (A), guanine (G), thymine (T), cytosine (C), or uracil (U)). In some embodiments, the linker (L) comprises one or more linker group, each linker group being independently selected from the group consisting of substituted or unsubstituted alkyl, substituted or unsubstituted heteroalkyl, and substituted or unsubstituted heteroaryl. In some embodiments, the linker (L) comprises one or more linker group, each linker group being independently selected from the group consisting of substituted or unsubstituted alkyl and substituted or unsubstituted heteroalkyl.

In some embodiments, the linker (L) is or comprises substituted or unsubstituted alkyl (e.g., straight or branched alkyl). In some embodiments, the substituted or unsubstituted alkyl is a straight alkyl, such as a straight C₁-C₁₅ alkyl (e.g., a straight C₁-C₁₀ alkyl (e.g., a straight C₃-C₈ alkyl)). In some embodiments, the substituted or unsubstituted alkyl is a branched alkyl, such as a branched C₁-C₁₅ alkyl (e.g., a branched C₁-C₁₀ alkyl (e.g., a branched C₃-C₈ alkyl)) having one or more branching carbon atoms (e.g., >CH—). In some embodiments, the linker (L) is unsubstituted alkyl.

In some embodiments, the linker (L) is (CH₂)ₙ, where n is 1-20. In some embodiments, n is 1-15. In some embodiments, n is 1-10. In some embodiments, n is 1-5. In some embodiments, n is 5-15 (e.g., n is 6 or 12).

In some embodiments, the linker (L) is or comprises substituted or unsubstituted heteroalkyl. In some embodiments, the substituted or unsubstituted heteroalkyl is a straight heteroalkyl, such as a straight C₁-C₁₅ heteroalkyl (e.g., a straight C₁-C₁₀ heteroalkyl (e.g., a straight C₂-C₈ heteroalkyl)). In some embodiments, the substituted or unsubstituted heteroalkyl is a branched heteroalkyl, such as a branched C₁-C₁₅ heteroalkyl (e.g., a branched C₁-C₁₀ heteroalkyl (e.g., a branched C₂-C₈ heteroalkyl) having one or more branching atom (e.g., >CH— or >N—). In some embodiments, the linker (L) is unsubstituted heteroalkyl. In some embodiments, the linker (L) is substituted heteroalkyl. In some embodiments, the linker (L) is heteroalkyl substituted with one or more substituent selected from oxo, alkyl, and alkoxy.

In some embodiments, the linker (L) is or comprises substituted or unsubstituted alkoxy. In some embodiments, the linker (L) is unsubstituted alkoxy.

In some embodiments, the linker (L) is or comprises —(CH₂CH₂O)ₘ—, —(OCH₂CH₂)ₘ—, —(CH₂CH₂O)ₘCH₂CH₂—, or —CH₂CH₂(OCH₂CH₂)ₘ—, Where m is 1-20. In some embodiments, m is 1-15. In some embodiments, m is 1-10. In some embodiments, m is 1-5. In some embodiments, m is 5-15 (e.g., m is 6 or 12).

In some embodiments, the linker (L) is or comprises —S(CH₂)ₒ—, —(CH₂)ₒS—, —SS(CH₂)ₒ—, or —(CH₂)ₒS—, where o is 1-20. In some embodiments, o is 1-15. In some embodiments, o is 1-10. In some embodiments, o is 1-5. In some embodiments, o is 5-15 (e.g., o is 6 or 12).

In some embodiments, a linker described herein (e.g., L) is a heteroalkyl linker (e.g., a linker that is or comprises a substituted or unsubstituted heteroalkyl). In some embodiments, the heteroalkyl linker comprises one or more heteroalkyl linker group, each heteroalkyl linker group being selected from the group consisting of O, S, alkoxy, amino, alkyl, amide, oxo, and hydroxy. In some embodiments, the heteroalkyl linker comprises one or more heteroalkyl linker group, each heteroalkyl linker group being selected from the group consisting of O, S, alkoxy, and alkyl. In some embodiments, the heteroalkyl linker comprises S and one or more alkyl group. In some embodiments, the heteroalkyl linker comprises O and one or more alkyl group. In some embodiments, the heteroalkyl linker is or comprises alkoxy (e.g., comprising one or more ethylene glycol). In some embodiments, the heteroalkyl linker is alkoxy (e.g., comprising one or more ethylene glycol). In some embodiments, the heteroalkyl linker comprises one or more modified or unmodified ribose and/or one or more modified or unmodified adenine (A), guanine (G), thymine (T), cytosine (C), or uracil (U). In some embodiments, the heteroalkyl linker comprises one or more nucleotide or nucleoside.

In some embodiments, the linker is or comprises substituted or unsubstituted heterocyclyl. In some embodiments, the linker comprises one or more modified or unmodified ribose.

In some embodiments, the linker is or comprises substituted or unsubstituted heteroaryl. In some embodiments, the linker comprises one or more modified or unmodified adenine (A), guanine (G), thymine (T), cytosine (C), or uracil (U).

In some embodiments, the linker comprises one or more nucleoside (e.g., adenosine). In some embodiments, the linker comprises one or more nucleotide (e.g., adenosine monophosphate or adenosine triphosphate).

Exemplary linkers (e.g., L) are provided in Table 4

TABLE 4

| Linker Example | Structure |
|---|---|
| 1 | —CH₂CH₂OCH₂CH₂— |
| 2 | —CH₂CH₂(OCH₂CH₂)₂— |
| 3 | —CH₂CH₂(OCH₂CH₂)₃— |
| 4 | —(OCH₂CH₂)₂CH₂CH₂— |
| 5 | —(OCH₂CH₂)₃CH₂CH₂— |
| 6 | CH₂CH₂ |
| 7 | —(CH₂CH₂)₃— |
| 8 | —(CH₂CH₂)₆— |
| 9 | 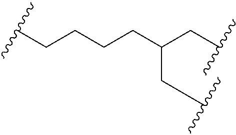 [SMILES: *CCCCC(C*)C*] |
| 10 | —S(CH₂CH₂)₃— |
| 11 | —SS(CH₂CH₂)₃— |
| 12 | —(CH₂CH₂)₃S— |
| 13 | —(CH₂CH₂)₃SS— |
| 14 | —S(CH₂CH₂)₆— |
| 15 | —SS(CH₂CH₂)₆— |
| 16 | —(CH₂CH₂)₆S— |
| 17 | —(CH₂CH₂)₆SS— |
| 18 | 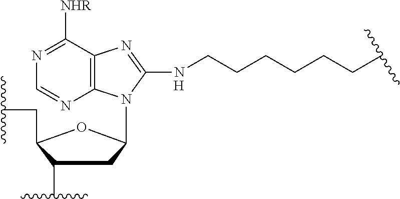 |
| | wherein R is H or a protecting group, such |
| | as benzyol (Bz). |

In some embodiments, an amine-modified nucleotide described herein (e.g., a first amine-modified nucleotide described herein, such as X) is linked to a linker described herein (e.g., L) through a linkage comprising an amide linkage. In some embodiments, an amine-modified nucleotide described herein (e.g., a second amine-modified nucleotide or a thiol-modified nucleotide described herein, such as Y) is linked to a linker described herein (e.g., L) through a linkage comprising an amide linkage or a thiol linkage.

In some embodiments, a linker described herein (e.g., L) is provided (e.g., incorporated into a polyA nucleotide described herein) using an amino modifier or a thio modifier. In some embodiments, a linker described herein (e.g., L) is provided using a 5′ amino modifier, a 5′-thio modifier, or a 3′-amino modifier. In some embodiments, a modifier described herein is used to introduce (e.g., at the 5′-terminal) a 5′-amine or thiol modification at adenosine in a (synthetic) poly(A) strand described herein. In some embodiments, linkers comprising a functionalizing COOH, such as linkers having maleimide or NHS ester at the 3′ terminal, are used to conjugate a modifier described herein to an amine from base-modified dA amines in poly(A) strands described herein. In some embodiments, the linker is used to incorporate a 5′-amine or thiol moiety into a short poly(A) strand at the 5′ end of a polynucleotide described herein. In some embodiments, a branching structure is formed with a poly(A) fragment containing a C8 base-modified dA. In some embodiments, a 3′ modifier described herein is used to reverse the linkage of a poly(A) chain described herein (e.g., from 3′ to 5′). In some embodiments, the linkage of a poly(A) chain described herein is reversed using a solid-phase poly(A) synthesis, such as using a 3′-amino modifier described herein (e.g., after deblocking and purification, the 3′ amine is utilized for further conjugation and the use of a base-modified dA provides inverted dA at 3′ end).

Exemplary amino and thio modifiers are provided in Table 5.

TABLE 5

| Modifier | |
|---|---|
| Example | Structure |
| 1 | 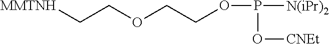 |
| 2 | 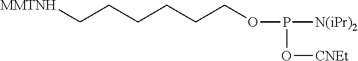 |
| 3 | 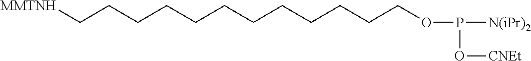 |
| 4 | 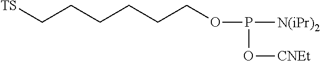 |
| 5 | 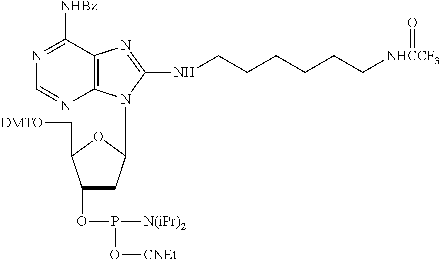 |
| 6 | 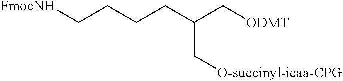 |

Modified mRNAs

In some embodiments, provided herein are modified ribonucleic acid molecules (e.g., mRNA) comprising, 5′ to 3, a 5′ cap, a region encoding a polypeptide, and the multi-terminus polynucleotides described herein. In certain embodiments, the modified ribonucleic acid molecules (e.g., mRNA) further comprise a 5′ UTR and a 3′ UTR. In certain embodiments, the multi-terminus polynucleotides comprises a first polyA polynucleotide linked to a second polyA polynucleotide, wherein the multi-terminus polyA polynucleotide comprises a formula represented by the structure:

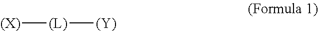

wherein:
   (X) is a first functionalized nucleotide within the first polyA polynucleotide, wherein the functionalized nucleotide comprises an amino modification, an alkoxyamino modification, a thiol modification, a carboxyl modification, or an aldehyde modification;
   (L) is a linker;
   (Y) is a second functionalized nucleotide within the second polyA polynucleotide, wherein the functionalized nucleotide comprises an amino modification, an alkoxyamino modification, a carboxyl modification, or an aldehyde modification;
   the first functionalized nucleotide is linked to the linker through a linkage comprising an amide linkage, an oxime linkage, a thioether linkage, and
   the linker is linked to the second functionalized nucleotide is linked to the linker through a linkage comprising an amide linkage, an oxime linkage, a thioether linkage.

In some embodiments, the multi-terminus polyA polynucleotide comprises a formula represented by the structure:

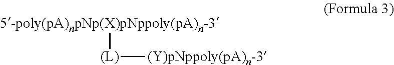

wherein:
   (X) is a first functionalized nucleotide within a first polyA polynucleotide, wherein the functionalized nucleotide comprises an amino modification, an alkoxyamino modification, a thiol modification, a carboxyl modification, or an aldehyde modification;
   (L) is a linker;
   (Y) is a second functionalized nucleotide at the 5′ position of a second polyA polynucleotide, wherein the functionalized nucleotide comprises an amino modification, an alkoxyamino modification, a carboxyl modification, or an aldehyde modification;
   each (p) is independently an inter-nucleoside linkage (e.g., phosphodiester or phosphorothioate bond) or terminal phosphate or phosphorothioate;
   each (N) is independently a nucleotide or a modified nucleotide;
   each (A) is independently an adenine nucleotide or a modified adenine nucleotide;
   each (n) is independently an integer equal to or greater than 1;
   the first functionalized nucleotide is linked to the linker through a linkage comprising an amide linkage, an oxime linkage, a thioether linkage, and
   the linker is linked to the functionalized nucleotide is linked to the linker through a linkage comprising an amide linkage, a oxime linkage, a thioether linkage.
      In such embodiments, the polarity of the second polyA polynucleotide is 5′→3′ (proximal to distal) relative to its linkages to (X) through (L).

In some embodiments, the multi-terminus polyA polynucleotide comprises a formula represented by the structure:

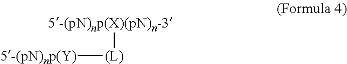

wherein:
   (X) is a first functionalized nucleotide within a first polyA polynucleotide, wherein the functionalized nucleotide comprises an amino modification, an alkoxyamino modification, a thiol modification, a carboxyl modification, or an aldehyde modification;
   (L) is a linker;
   (Y) is a second functionalized nucleotide at the 3′ position of a second polyA polynucleotide, wherein the functionalized nucleotide comprises an amino modification, an alkoxyamino modification, a carboxyl modification, or an aldehyde modification;
   each (p) is independently an inter-nucleoside linkage (e.g., phosphodiester or phosphorothioate bond) or terminal phosphate or phosphorothioate;
   each (N) is independently a nucleotide or a modified nucleotide;
   each (n) is independently an integer equal to or greater than 1;
   the first functionalized nucleotide is linked to the linker (e.g., through a linkage comprising an amide linkage, an oxime linkage, a thioether linkage); and
   the linker is linked to the second functionalized nucleotide (e.g., through a linkage comprising an amide linkage, an oxime linkage, a thioether linkage). In such embodiments, the polarity of the second polyA polynucleotide is 3′→5′ (proximal to distal) relative to its linkages to (X) through (L).
      In some embodiments, the multi-terminus polyA polynucleotide comprises a formula represented by the structure:

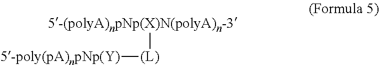

wherein:
   (X) is a first functionalized nucleotide within a first polyA polynucleotide, wherein the functionalized nucleotide comprises an amino modification, an alkoxyamino modification, a thiol modification, a carboxyl modification, or an aldehyde modification;
   (L) is a linker;
   (Y) is a second functionalized nucleotide at the 3′ position of a second polyA polynucleotide, wherein the functionalized nucleotide comprises an amino modification, an alkoxyamino modification, a carboxyl modification, or an aldehyde modification;
   each (p) is independently an inter-nucleoside linkage (e.g., phosphodiester or phosphorothioate bond) or terminal phosphate or thiophosphate;
   each (N) is independently a nucleotide or a modified nucleotide;
   each (A) is independently an adenine nucleotide or a modified adenine nucleotide;
   each (n) is independently an integer equal to or greater than 1;
   the first functionalized nucleotide is linked to the linker (e.g., through a linkage comprising an amide linkage, an oxime linkage, a thioether linkage), and
   the linker is linked to the second functionalized nucleotide is linked to the linker (e.g., through a linkage comprising an amide linkage, an oxime linkage, a thioether linkage).
      In such embodiments, the polarity of the second polyA polynucleotide is 3′→5′ (proximal to distal) relative to its linkages to (X) through (L).

In some embodiments, the multi-terminus polyA polynucleotide comprises a formula represented by the structure:

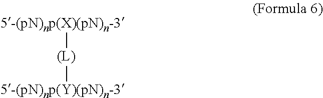

wherein:
   (X) is a first functionalized nucleotide within a first polyA polynucleotide, wherein the functionalized nucleotide comprises an amino modification, an alkoxyamino modification, a thiol modification, a carboxyl modification, or an aldehyde modification;
   (L) is a linker;
   (Y) is a second functionalized nucleotide within a second polyA polynucleotide, wherein the functionalized nucleotide comprises an amino modification, an alkoxyamino modification, a carboxyl modification, or an aldehyde modification;
   each (p) is independently an inter-nucleoside linkage (e.g., phosphodiester or phosphorothioate bond) or terminal phosphate or phosphorothioate;
   each (N) is independently a nucleotide or a modified nucleotide;
   each (n) is independently an integer equal to or greater than 1;
   the first functionalized nucleotide is linked to the linker through a linkage comprising an amide linkage, an oxime linkage, a thioether linkage, and
   the linker is linked to the second functionalized nucleotide through a linkage comprising an amide linkage, a oxime linkage, a thioether linkage.

In some embodiments, the multi-terminus polyA polynucleotide comprises a formula represented by the structure:

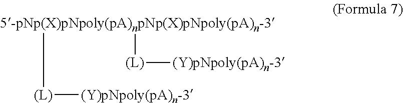

wherein:
   each (X) is independently a first functionalized nucleotide within a first polyA polynucleotide, wherein the functionalized nucleotide comprises an amino modification, an alkoxyamino modification, a thiol modification, a carboxyl modification, or an aldehyde modification;
   each (L) is independently a linker;
   each (Y) is independently a second functionalized nucleotide at the 5′ position of a second polyA polynucleotide, wherein the functionalized nucleotide comprises an amino modification, an alkoxyamino modification, a carboxyl modification, or an aldehyde modification;
   each (p) is independently an inter-nucleoside linkage (e.g., phosphodiester or phosphorothioate bond) or terminal phosphate or phosphorothioate;
   each (N) is independently a nucleotide or a modified nucleotide;
   each (A) is independently an adenine nucleotide or a modified adenine nucleotide;
   each (n) is independently an integer equal to or greater than 1;
   the first functionalized nucleotide is linked to the linker through a linkage comprising an amide linkage, an oxime linkage, a thioether linkage, and
   the linker is linked to the second functionalized nucleotide through a linkage comprising an amide linkage, a oxime linkage, a thioether linkage.
      In such embodiments, the polarity of the second polyA polynucleotide is 5′→3′ (proximal to distal) relative to its linkages to (X) through (L).

In some embodiments, the multi-terminus polyA polynucleotide comprises a formula represented by the structure:

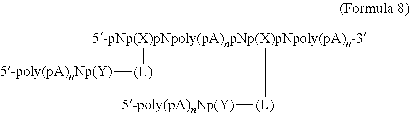

wherein:
   each (X) is independently a first functionalized nucleotide within a first polyA polynucleotide, wherein the functionalized nucleotide comprises an amino modification, an alkoxyamino modification, a thiol modification, a carboxyl modification, or an aldehyde modification;
   each (L) is independently a linker;
   each (Y) is independently a second functionalized nucleotide at the 3′ position of a second polyA polynucleotide, wherein the functionalized nucleotide comprises an amino modification, an alkoxyamino modification, a carboxyl modification, or an aldehyde modification;
   each (p) is independently an inter-nucleoside linkage (e.g., phosphodiester or phosphorothioate bond) or terminal phosphate or phosphorothioate;
   each (N) is independently a nucleotide or a modified nucleotide;
   each (A) is independently an adenine nucleotide or a modified adenine nucleotide;
   each (n) is independently an integer equal to or greater than 1;
   the first functionalized nucleotide is linked to the linker (e.g., through a linkage comprising an amide linkage, an oxime linkage, a thioether linkage); and
   the linker is linked to the second functionalized nucleotide (e.g., through a linkage comprising an amide linkage, an oxime linkage, a thioether linkage).

In some embodiments, the modified ribonucleic acid molecule further comprises a 5′ UTR located 3′ to the 5′ cap and 5′ to the region encoding the polypeptide. In some embodiments, the modified ribonucleic acid molecule further comprises a 3′ UTR located 3′ to the region encoding the polypeptide and 5′ to multi-terminus polyA polynucleotide.

### Compositions Substantially Free of Copper

In certain instances, copper is toxic to cells. For example, even minute quantities can lead to cellular damage or death. In related instances, removing residual copper from click chemistry reactions can be challenging due to its strong affinity for proteins and organic molecules. Additionally, the complex nature of the reaction system and because copper ions can be highly dispersed and tightly bound within the product mixture make its complete removal difficult.

In some embodiments, provided are compositions of multi-terminus polynucleotides (e.g., multi-terminus mRNAs) comprising a first polyA polynucleotide linked to a second polyA polynucleotide, wherein the composition is substantially free of copper). In certain embodiments, the multi-terminus polynucleotides comprise a formula represented by the structures in any one of Formulas 1-8. In such embodiments, the first and the second functionalized nucleotide comprise an amino modification, an alkoxyamino modification, a thiol modification, a carboxyl modification, an aldehyde modification, an azide modification, or an alkyne modification. Also in such embodiments, first functionalized nucleotide is linked to the linker through a linkage comprising an amide linkage, an oxime linkage, a triazole linkage, or a thioether linkage, and second functionalized nucleotide is linked to the linker through a linkage comprising an amide linkage, an oxime linkage, a triazole linkage, or a thioether linkage.

The amount of copper (e.g., trace copper) in a solution can be measured by inductively coupled plasma mass spectrometry (ICP-MS). In certain embodiments, the composition is substantially free of trace copper. In certain embodiments, the composition comprises less than 50 parts per million (ppm) copper. In certain embodiments, the composition comprises less than 25 parts per million (ppm) copper. In certain embodiments, the composition comprises less than 10 parts per million (ppm) copper. In certain embodiments, the composition comprises less than 5 parts per million (ppm) copper. In certain embodiments, the composition comprises less than 1 parts per million (ppm) copper. In certain embodiments, the composition comprises less than 0.1 parts per million (ppm) copper. In certain embodiments, the composition comprises less than 0.05 parts per million (ppm) copper. In certain embodiments, the composition does not comprise copper.

### Methods

In some embodiments, provided are methods of increasing translation of a polypeptide, the method comprising: contacting a cell with (i) a messenger ribonucleic acid molecule comprising the multi-terminus polyA polynucleotides described herein or (ii) the modified ribonucleic acid molecules described herein.

In some embodiments, increasing translation comprises increasing the total amount of the polypeptide. In some embodiments, increasing translation comprises increasing the duration of protein translation.

In some embodiments, provided are methods of increasing the half-life of a ribonucleic acid molecule, the method comprising: attaching the multi-terminus polynucleotides described herein the 3′ end of the ribonucleic acid molecule. In certain embodiments, the ribonucleic acid molecule is a messenger ribonucleic acid (mRNA) molecule. In certain embodiments, the mRNA molecule comprises, 5′ to 3, a 5′ cap, a 5′ UTR, a region encoding a polypeptide, and a 3′ UTR.

As used herein, “a” or “an” when used in conjunction with the term “comprising” in the claims and/or the specification includes and/or refers to “one” and also consistent with the meaning of “one or more”, “at least one”, and “one or more than one”. Similarly, the word “another” may mean at least a second or more.

As used herein, the words “comprising” (and any form of comprising, such as “comprise” and “comprises”), “having” (and any form of having, such as “have” and “has”), “including” (and any form of including, such as “include” and “includes”) or “containing” (and any form of containing, such as “contain” and “contains”), are inclusive or open-ended and do not exclude additional, unrecited elements or process steps. As used herein, in any instance or embodiment described herein, “comprising” may be replaced with “consisting essentially of” and/or “consisting of”. used herein, in any instance or embodiment described herein, “comprises” may be replaced with “consists essentially of” and/or “consists of”.

As used herein, the term “about” in the context of a given value or range includes and/or refers to a value or range that is within 20%, within 10%, and/or within 5% of the given value or range.

As used herein, the term “and/or” is to be taken as specific disclosure of each of the two specified features or components with or without the other. For example, “A and/or B” is to be taken as specific disclosure of each of (i) A, (ii) B and (iii) A and B, just as if each were set out individually herein.

As used herein, a “sample” includes and/or refers to any fluid or liquid sample which is being analyzed in order to detect and/or quantify an analyte. In some embodiments, a sample is a biological sample. Examples of samples include without limitation a bodily fluid, an extract, a solution containing proteins and/or DNA, a cell extract, a cell lysate, or a tissue lysate. Non-limiting examples of bodily fluids include urine, saliva, blood, serum, plasma, cerebrospinal fluid, tears, semen, sweat, pleural effusion, liquified fecal matter, and lacrimal gland secretion.

“Amino” refers to the —NH₂ radical.

“Cyano” refers to the —CN radical.

“Nitro” refers to the —NO₂ radical.

“Oxo” refers to the ═O radical.

“Hydroxyl” refers to the —OH radical.

“Alkyl” generally refers to an acyclic (e.g., straight or branched) or cyclic hydrocarbon (e.g., chain) radical consisting solely of carbon and hydrogen atoms, such as having from one to fifteen carbon atoms (e.g., C₁-C₁₅ alkyl). Unless otherwise state, alkyl is saturated or unsaturated (e.g., an alkenyl, which comprises at least one carbon-carbon double bond). Disclosures provided herein of an “alkyl” are intended to include independent recitations of a saturated “alkyl,” unless otherwise stated. Alkyl groups described herein are generally monovalent, but may also be divalent (which may also be described herein as “alkylene” or “alkylenyl” groups). In certain embodiments, an alkyl comprises one to thirteen carbon atoms (e.g., C₁-C₁₃ alkyl). In certain embodiments, an alkyl comprises one to eight carbon atoms (e.g., C₁-C₈ alkyl). In other embodiments, an alkyl comprises one to five carbon atoms (e.g., C₁-C₅ alkyl). In other embodiments, an alkyl comprises one to four carbon atoms (e.g., C₁-C₄ alkyl). In other embodiments, an alkyl comprises one to three carbon atoms (e.g., C₁-C₃ alkyl). In other embodiments, an alkyl comprises one to two carbon atoms (e.g., C₁-C₂ alkyl). In other embodiments, an alkyl comprises one carbon atom (e.g., C₁ alkyl). In other embodiments, an alkyl comprises five to fifteen carbon atoms (e.g., C₅-C₁₅ alkyl). In other embodiments, an alkyl comprises five to eight carbon atoms (e.g., C₅-C₈ alkyl). In other embodiments, an alkyl comprises two to five carbon atoms (e.g., C₂-C₅ alkyl). In other embodiments, an alkyl comprises three to five carbon atoms (e.g., C₃-C₅ alkyl). In other embodiments, the alkyl group is selected from methyl, ethyl, 1-propyl (n-propyl), 1-methylethyl (iso-propyl), 1-butyl (n-butyl), 1-methylpropyl (sec-butyl), 2-methylpropyl (iso-butyl), 1,1-dimethylethyl (tert-butyl), 1-pentyl (n-pentyl). The alkyl is attached to the rest of the molecule by a single bond. In general, alkyl groups are each independently substituted or unsubstituted. In some instances, an alkyl group provided herein is substituted or further substituted by one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})S(O)ₜR^{a} (where t is 1 or 2), —S(O)ₜOR^{a} (where t is 1 or 2), —S(O)ₜR^{a} (where t is 1 or 2) and —S(O)ₜN(R^{a})₂ (where t is 1 or 2) where each R^{a} is independently hydrogen, alkyl (optionally substituted with halogen, hydroxy, methoxy, or trifluoromethyl), fluoroalkyl, carbocyclyl (optionally substituted with halogen, hydroxy, methoxy, or trifluoromethyl), carbocyclylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl).

“Alkoxy” refers to a radical bonded through an oxygen atom of the formula-O-alkyl, where alkyl is an alkyl group as defined above.

“Alkenyl” refers to a straight or branched hydrocarbon chain radical group consisting solely of carbon and hydrogen atoms, containing at least one carbon-carbon double bond, and having from two to twelve carbon atoms. In certain embodiments, an alkenyl comprises two to eight carbon atoms. In other embodiments, an alkenyl comprises two to four carbon atoms. Unless stated otherwise specifically in the specification, an alkenyl group provided herein is substituted or further substituted as described for “alkyl” hereinabove.

“Alkylene” or “alkylene chain” generally refers to a straight or branched divalent alkyl group linking the rest of the molecule to a radical group, such as having from one to twelve carbon atoms, for example, methylene, ethylene, propylene, i-propylene, n-butylene, and the like. Unless stated otherwise specifically in the specification, an alkylene chain is substituted or further substituted as described for “alkyl” hereinabove.

“Aryl” refers to a radical derived from an aromatic monocyclic or multicyclic hydrocarbon ring system by removing a hydrogen atom from a ring carbon atom. The aromatic monocyclic or multicyclic hydrocarbon ring system contains only hydrogen and carbon from five to eighteen carbon atoms, where at least one of the rings in the ring system is fully unsaturated, i.e., it contains a cyclic, delocalized (4n+2) π-electron system in accordance with the Hückel theory. The ring system from which aryl groups are derived include, but are not limited to, groups such as benzene, fluorene, indane, indene, tetralin and naphthalene. In some instances, an aryl group provided herein is substituted or further substituted by one or more substituents independently selected from alkyl, alkenyl, alkynyl, halo, fluoroalkyl, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a}) 2 (where t is 1 or 2), where each R^{a} is independently hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl), each R^{b} is independently a direct bond or a straight or branched alkylene or alkenylene chain, and R^{c} is a straight or branched alkylene or alkenylene chain, and where each of the above substituents is unsubstituted unless otherwise indicated.

“Carbocyclyl” or “cycloalkyl” refers to a stable non-aromatic monocyclic or polycyclic hydrocarbon radical consisting solely of carbon and hydrogen atoms, which includes fused or bridged ring systems, having from three to fifteen carbon atoms. In certain embodiments, a carbocyclyl comprises three to ten carbon atoms. In other embodiments, a carbocyclyl comprises five to seven carbon atoms. The carbocyclyl is attached to the rest of the molecule by a single bond. Carbocyclyl or cycloalkyl is saturated (i.e., containing single C—C bonds only) or unsaturated (i.e., containing one or more double bonds or triple bonds). Examples of saturated cycloalkyls include, e.g., cyclopropyl, cyclobutyl, cyclopentyl, cyclohexyl, cycloheptyl, and cyclooctyl. An unsaturated carbocyclyl is also referred to as “cycloalkenyl.” Examples of monocyclic cycloalkenyls include, e.g., cyclopentenyl, cyclohexenyl, cycloheptenyl, and cyclooctenyl. Polycyclic carbocyclyl radicals include, for example, adamantyl, norbornyl (i.e., bicyclo[2.2.1]heptanyl), norbornenyl, decalinyl, 7,7-dimethyl-bicyclo[2.2.1]heptanyl, and the like. In some instances, a carbocyclyl group provided herein is substituted or further substituted with one or more substituents independently selected from alkyl, alkenyl, alkynyl, halo, fluoroalkyl, oxo, thioxo, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2), where each R^{a} is independently hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl), each R^{b} is independently a direct bond or a straight or branched alkylene or alkenylene chain, and R^{c} is a straight or branched alkylene or alkenylene chain, and where each of the above substituents is unsubstituted unless otherwise indicated.

“Halo” or “halogen” refers to fluoro, bromo, chloro, or iodo substituents.

“Haloalkyl” refers to an alkyl radical, as defined above, that is substituted by one or more halogen radicals, as defined above, for example, trihalomethyl, dihalomethyl, halomethyl, and the like. In some embodiments, the haloalkyl is a fluoroalkyl, such as, for example, trifluoromethyl, difluoromethyl, fluoromethyl, 2,2,2-trifluoroethyl, 1-fluoromethyl-2-fluoroethyl, and the like. In some embodiments, the alkyl part of the fluoroalkyl radical is substituted or further substituted as defined above for an alkyl group.

The term “heteroalkyl” refers to an alkyl group as defined above in which one or more skeletal carbon atoms of the alkyl are substituted with a heteroatom (with the appropriate number of substituents or valencies—for example, —CH₂— may be replaced with —NH— or —O—). For example, each substituted carbon atom is independently substituted with a heteroatom, such as wherein the carbon is substituted with a nitrogen, oxygen, sulfur, or other suitable heteroatom. In some instances, each substituted carbon atom is independently substituted for an oxygen, nitrogen (e.g. —NH—, —N(alkyl)-, or —N(aryl)- or having another substituent contemplated herein), or sulfur (e.g. —S—, —S(═O)—, or —S(═O)₂—). In some embodiments, a heteroalkyl is attached to the rest of the molecule at a carbon atom of the heteroalkyl. In some embodiments, a heteroalkyl is attached to the rest of the molecule at a heteroatom of the heteroalkyl. In some embodiments, a heteroalkyl is a C₁-C₁₈ heteroalkyl. In some embodiments, a heteroalkyl is a C₁-C₁₂ heteroalkyl. In some embodiments, a heteroalkyl is a C₁-C₆ heteroalkyl. In some embodiments, a heteroalkyl is a C₁-C₄ heteroalkyl. In some embodiments, heteroalkyl includes alkylamino, alkylaminoalkyl, aminoalkyl, heterocycloalkyl, heterocycloalkyl, heterocyclyl, and heterocycloalkylalkyl, as defined herein. Unless stated otherwise specifically in the specification, heteroalkyl does not include alkoxy as defined herein. Unless stated specifically otherwise, a heteroalkyl group is substituted or further substituted as defined above for an alkyl group.

“Heteroalkylene” refers to a divalent heteroalkyl group defined above which links one part of the molecule to another part of the molecule. Unless stated specifically otherwise, a heteroalkylene is substituted or further substituted, as defined above for an alkyl group.

“Heterocyclyl” refers to a stable 3- to 18-membered non-aromatic ring radical that comprises two to twelve carbon atoms and from one to six heteroatoms selected from nitrogen, oxygen and sulfur. Unless stated otherwise specifically in the specification, the heterocyclyl radical is a monocyclic, bicyclic, tricyclic or tetracyclic ring system, which optionally includes fused or bridged ring systems. The heteroatoms in the heterocyclyl radical are optionally oxidized. One or more nitrogen atoms, if present, are optionally quaternized. The heterocyclyl radical is partially or fully saturated. The heterocyclyl radical is saturated (i.e., containing single C—C bonds only) or unsaturated (e.g., containing one or more double bonds or triple bonds in the ring system). In some instances, the heterocyclyl radical is saturated. In some instances, the heterocyclyl radical is saturated and substituted. In some instances, the heterocyclyl radical is unsaturated. Examples of such heterocyclyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. In some instances, a heterocyclyl group is substituted or further substituted with one or more substituents selected from alkyl, alkenyl, alkynyl, halo, fluoroalkyl, oxo, thioxo, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2), where each R^{a} is independently hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl), each R^{b} is independently a direct bond or a straight or branched alkylene or alkenylene chain, and R^{c} is a straight or branched alkylene or alkenylene chain, and where each of the above substituents is unsubstituted unless otherwise indicated.

“Heteroaryl” refers to a radical derived from a 3- to 18-membered aromatic ring radical that comprises two to seventeen carbon atoms and from one to six heteroatoms selected from nitrogen, oxygen and sulfur. As used herein, the heteroaryl radical is a monocyclic, bicyclic, tricyclic or tetracyclic ring system, wherein at least one of the rings in the ring system is fully unsaturated, i.e., it contains a cyclic, delocalized (4n+2) π-electron system in accordance with the Hückel theory. Heteroaryl includes fused or bridged ring systems. The heteroatom(s) in the heteroaryl radical is optionally oxidized. One or more nitrogen atoms, if present, are optionally quaternized. The heteroaryl is attached to the rest of the molecule through any atom of the ring(s). Examples of heteroaryls include, but are not limited to, azepinyl, acridinyl, benzimidazolyl, benzindolyl, 1,3-benzodioxolyl, benzofuranyl, benzooxazolyl, benzo[d]thiazolyl, benzothiadiazolyl, benzo[b][1,4]dioxepinyl, benzo[b][1,4]oxazinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzothieno[3,2-d]pyrimidinyl, benzotriazolyl, benzo[4,6]imidazo[1,2-a]pyridinyl, carbazolyl, cinnolinyl, cyclopenta[d]pyrimidinyl, 6,7-dihydro-5H-cyclopenta[4,5]thieno[2,3-d]pyrimidinyl, 5,6-dihydrobenzo[h]quinazolinyl, 5,6-dihydrobenzo[h]cinnolinyl, 6,7-dihydro-5H-benzo[6,7]cyclohepta[1,2-c]pyridazinyl, dibenzofuranyl, dibenzothiophenyl, furanyl, furanonyl, furo[3,2-c]pyridinyl, 5,6,7,8,9,10-hexahydrocycloocta[d]pyrimidinyl, 5,6,7,8,9,10-hexahydrocycloocta[d]pyridazinyl, 5,6,7,8,9,10-hexahydrocycloocta[d]pyridinyl, isothiazolyl, imidazolyl, indazolyl, indolyl, indazolyl, isoindolyl, indolinyl, isoindolinyl, isoquinolyl, indolizinyl, isoxazolyl, 5,8-methano-5,6,7,8-tetrahydroquinazolinyl, naphthyridinyl, 1,6-naphthyridinonyl, oxadiazolyl, 2-oxoazepinyl, oxazolyl, oxiranyl, 5,6,6a,7,8,9,10,10a-octahydrobenzo[h]quinazolinyl, 1-phenyl-1H-pyrrolyl, phenazinyl, phenothiazinyl, phenoxazinyl, phthalazinyl, pteridinyl, purinyl, pyrrolyl, pyrazolyl, pyrazolo[3,4-d]pyrimidinyl, pyridinyl, pyrido[3,2-d]pyrimidinyl, pyrido[3,4-d]pyrimidinyl, pyrazinyl, pyrimidinyl, pyridazinyl, pyrrolyl, quinazolinyl, quinoxalinyl, quinolinyl, isoquinolinyl, tetrahydroquinolinyl, 5,6,7,8-tetrahydroquinazolinyl, 5,6,7,8-tetrahydrobenzo[4,5]thieno[2,3-d]pyrimidinyl, 6,7,8,9-tetrahydro-5H-cyclohepta[4,5]thieno[2,3-d]pyrimidinyl, 5,6,7,8-tetrahydropyrido[4,5-c]pyridazinyl, thiazolyl, thiadiazolyl, triazolyl, tetrazolyl, triazinyl, thieno[2,3-d]pyrimidinyl, thieno[3,2-d]pyrimidinyl, thieno[2,3-c]pyridinyl, and thiophenyl (i.e. thienyl). In some instances, a heteroaryl group is substituted or further substituted with one or more substituents selected from alkyl, alkenyl, alkynyl, halo, fluoroalkyl, haloalkenyl, haloalkynyl, oxo, thioxo, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2), where each R^{a} is independently hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl), each R^{b} is independently a direct bond or a straight or branched alkylene or alkenylene chain, and R^{c} is a straight or branched alkylene or alkenylene chain, and where each of the above substituents is unsubstituted unless otherwise indicated.

In general, optionally substituted groups are each independently substituted or unsubstituted. Each recitation of a optionally substituted group provided herein, unless otherwise stated, includes an independent and explicit recitation of both an unsubstituted group and a substituted group (e.g., substituted in certain embodiments, and unsubstituted in certain other embodiments). In some instances, a substituted group provided herein (e.g., substituted alkyl) is substituted by one or more substituent, each substituent being independently selected from the group consisting of halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})S(O)ₜR^{a} (where t is 1 or 2), —S(O)ₜOR^{a} (where t is 1 or 2), —S(O)ₜR^{a} (where t is 1 or 2) and —S(O)ₜN(R^{a})₂ (where t is 1 or 2), where each R^{a} is independently hydrogen, alkyl (e.g., optionally substituted with halogen, hydroxy, methoxy, or trifluoromethyl), fluoroalkyl, carbocyclyl (e.g., optionally substituted with halogen, hydroxy, methoxy, or trifluoromethyl), carbocyclylalkyl (e.g., optionally substituted with halogen, hydroxy, methoxy, or trifluoromethyl), aryl (e.g., optionally substituted with halogen, hydroxy, methoxy, or trifluoromethyl), aralkyl (e.g., optionally substituted with halogen, hydroxy, methoxy, or trifluoromethyl), heterocyclyl (e.g., optionally substituted with halogen, hydroxy, methoxy, or trifluoromethyl), heterocyclylalkyl (e.g., optionally substituted with halogen, hydroxy, methoxy, or trifluoromethyl), heteroaryl (e.g., optionally substituted with halogen, hydroxy, methoxy, or trifluoromethyl), or heteroarylalkyl (e.g., optionally substituted with halogen, hydroxy, methoxy, or trifluoromethyl).

### EXAMPLES

### Example 1—Synthesis of Branched MOE-PS-RNA Oligonucleotides Using Amide-Based Linkages

### Synthesis of RNA Backbone MOE-PS Oligonucleotide

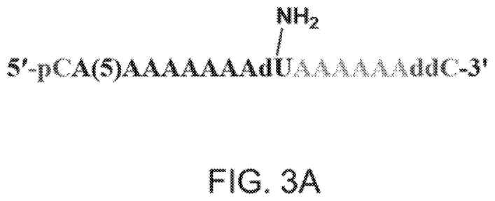

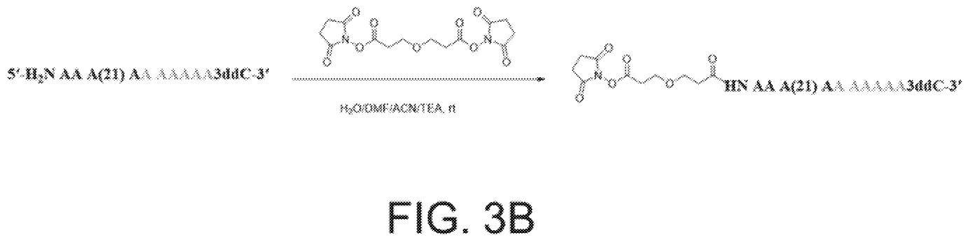

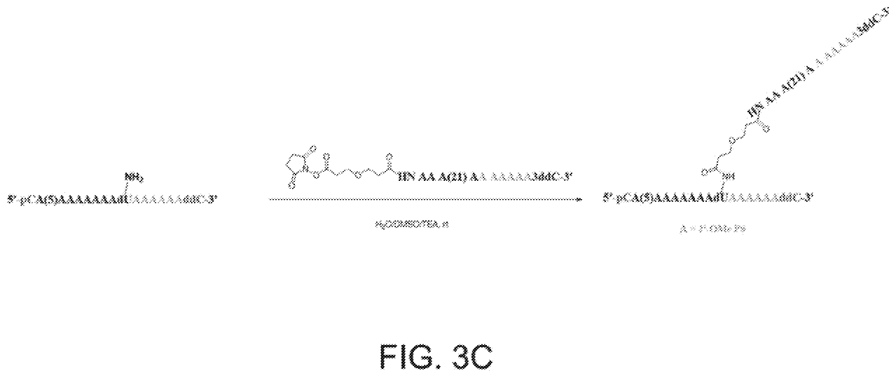

A branched strand 5′-amine MOE-PS modified oligonucleotides was synthesized through solid-phase synthesis using K&A H-8 at 4 μmol scale with MMT on at 5′ amine. The 0.1 M of phosphoramidite concentration was used during the synthesis. The cleavage and deprotection of synthesized oligonucleotides from the solid support was performed using AMA (1:1, 28% aqueous ammonia and 40% aqueous methylamine) at room temperature for 5 hours. AMA filtered (to remove solid support particles) and dried down on SP-Genevac. Dried oligonucleotides dissolved in dry DMSO and subjected to O-TBDMS deprotection of 2′-OH position at 65° C. for 2.5 hours. The deprotection reaction was quenched using 3M sodium acetate followed by ethanol precipitation to yield a solid material. At this stage oligos were purified using IEX chromatography based on purity. FIG. 3A shows an exemplary 5′-amine MOE-PS modified oligonucleotide.

Synthesized backbone oligonucleotides was purified through IEX chromatography using an TSKgel SuperQ-5 PW column (13 μm particle size, 150×21.5 mm, TOSCH Bioscience). Chromatographic purification was performed by using Mobile Phase A: 20 mM NaH2PO4 in 15% ACN; Mobile Phase B: 20 mM NaH2PO4 in 15% ACN+1M NaBr, Temp: 60° C., using the following gradient with a flow rate of 5 mL/min: 0% solvent B at 0 min followed by a linear gradient to 25% solvent B at 2 min, to 60% solvent B at 35 min, to 100% solvent B at 40 min. Oligonucleotides were detected at 260 nm. The pure fraction was collected and evaporated overnight using a SP-Genevac EZ-24.0, then re-dissolve the resulting solid in water. Purified oligonucleotides were desalted using a 3k amicon spin column to obtain the desired pure compound in (13.5 mg) isolated yield.

### Activated NHS Ester Modified Oligonucleotide Preparation

In a 5 mL falcon tube, 5′-amine-modified oligonucleotides (8.0 mg, 1 equiv.) was dissolved in water (500 μL). In a 5 mL falcon tube, NHS linker (66 mg, 200 equiv.) was dissolved in 500 μL of DMF. Then 500 μL of the linker solution was added to oligonucleotide solution together with 500 μL of acetonitrile and 10 μL of triethylamine. The mixture was shaken at room temperature for 30 minutes. After 30 minutes, precipitate the product by adding 3M NaOAc (0.6 mL) and absolute ethanol (5.4 mL) then keep it at −30° C. for 1 hour. Centrifuge the resulting solution and and decant the supernatant solution. The resulting solid was dissolved in 5 mL acetonitrile, and centrifuge the resulting suspension and decant the supernatant solution. The resulting crude material was directly used for the further amide coupling reaction. FIG. 3B shows a reaction schematic for preparing activated NHS ester modified oligonucleotide.

### Synthesis of Mono Branched MOE-PS Oligo Using Amide Coupling:

In a 5 mL falcon tube, branched oligonucleotides (10.89 mg, 2 equiv) was dissolved in 500 UL water. Then, activated NHS ester 5′-amine-modified oligonucleote (8 mg, 1 equiv.), 500 μL, DMSO, 500 μL acetonitrile, and 10 μL triethylamine were added. The resulting reaction mixture was shaken at room temperature overnight. After overnight reaction, precipitate the product by adding 3M NaOAc (0.6 mL) and absolute ethanol (5.4 mL) then keep it at −30° C. for 1 hour. Centrifuge the resulting solution and and decant the supernatant solution. The resulting crude solid was purified using IEX chromatography. FIG. 3C shows a reaction schematic.

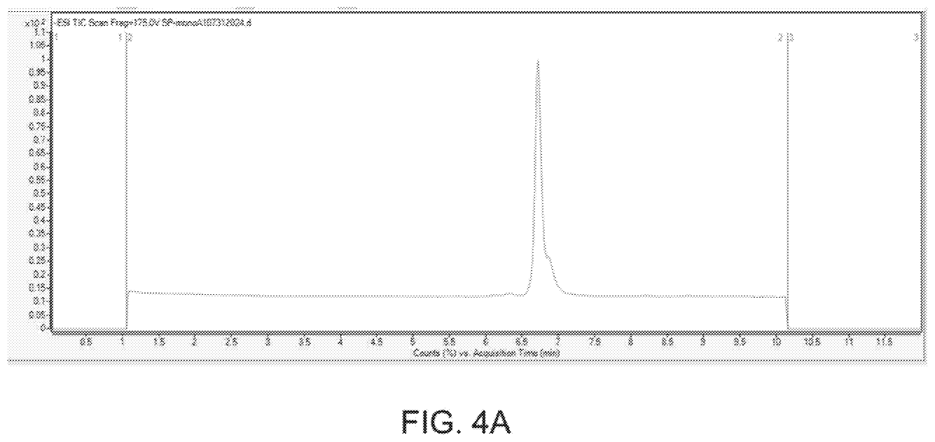

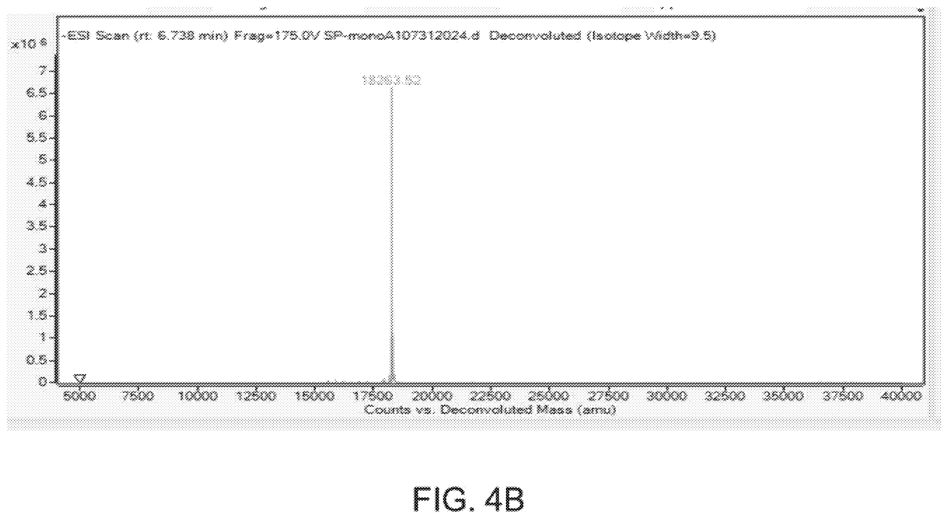

Synthesized backbone oligonucleotides was purified through IEX chromatography using an TSKgel SuperQ-5 PW column (13 μm particle size, 150×21.5 mm, TOSCH Bioscience). Chromatographic purification was performed by using Mobile Phase A: 20 mM NaH2PO4 in 15% ACN; Mobile Phase B: 20 mM NaH2PO4 in 15% ACN +1M NaBr, Temp: 60° C., using the following gradient with a flow rate of 5 mL/min: 0% solvent B at 0 min followed by a linear gradient to 25% solvent B at 2 min, to 60% solvent B at 35 min, to 100% solvent B at 40 min. Oligonucleotides were detected at 260 nm. The pure fraction was collected and evaporated overnight using a SP-Genevac EZ-24.0, then re-dissolve the resulting solid in water. Purified oligonucleotides were desalted using a 3k amicon spin column to obtain the desired pure compound in (3.5 mg) isolated yield. FIGS. 4A and 4B show LC/Q-TOF analysis and mass determination of mono branched MOE-PS oligo. LC/Q-TOF analysis confirmed the mass of mono branched MOE-PS oligo.

mRNAs Having Branched polyA Tails Using Amide Chemistries Improves mRNA Stability and Increased Protein Expression

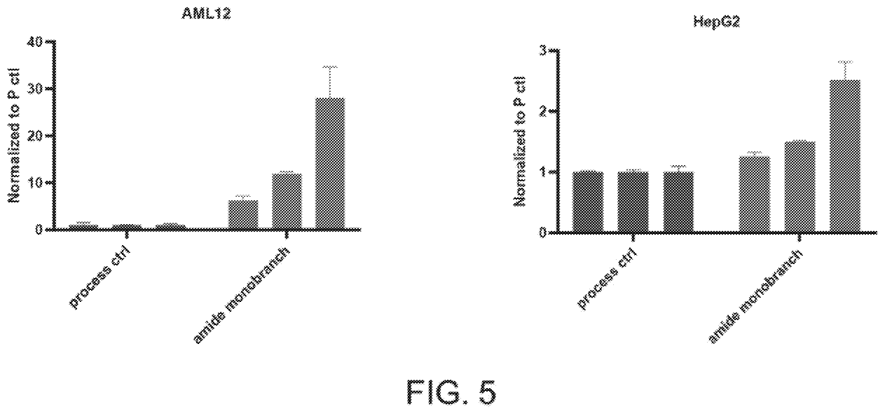

Translation stability of mRNA's having branched polyA tails using amide chemistries was evaluated using the cellular expression (AML12 cells and HepG2 cells) of the luciferase encoded by the mRNA. The luciferase activity was measured at day 1-3 after transfection with MessangerMax. The activity at each time point is normalized to process control (unligated mRNA) of the same time point. As demonstrated in FIG. 5, the increase of the normalized value from day 1-3 indicates an increased stability compared to the control mRNA.

### Example 2—Copper-Free Synthesis of Branched

### Synthesis of RNA Oligonucleotides

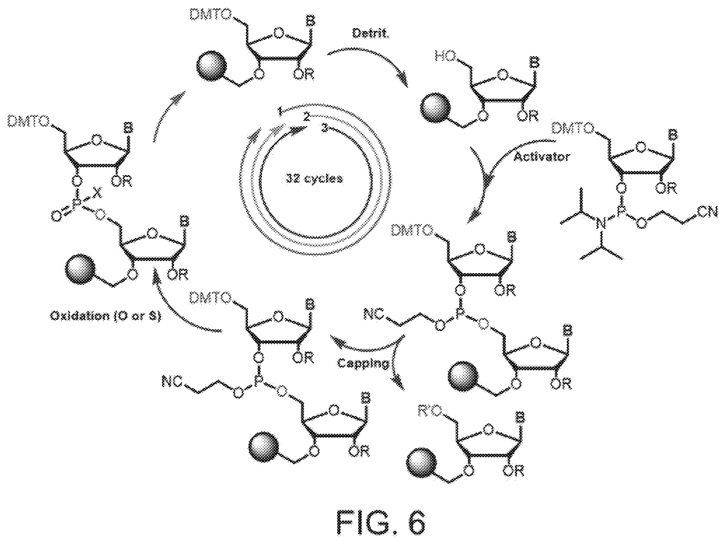

FIG. 6 shows a general schematic of oligo synthesis. RNA oligonucleotides were synthesized through solid-phase synthesis using a Sierra biosystems K&A H-8 or AKTA Oligosynt synthesizer. For the K&A synthesizer, oligos were synthesized on a 1 μmol scale. A phosphoramidite concentration of 0.12 M was used for all amidites. Deblock was performed using 3% DCA/DCM twice for 70 s. For coupling, 0.25-0.35 M ETT in ACN was employed as the activator using a 6 minute coupling time. For all PS amidites, double couplings were performed to ensure high coupling efficiency. Capping, oxidation, and sulfurization were performed using standard reagents using the manufacturer default protocols.

For the Oligosynth, a phosphoramidite concentration of 0.1M was used during the synthesis. Deblock was performed using 3% DCA/DCM once for 2.5 minutes. For coupling, 0.25-0.35 M ETT in ACN was employed as the activator using a 12 minute coupling time with recirculation. Capping, oxidation, and sulfurization were performed using standard reagents using the manufacturer default protocols.

The cleavage and deprotection of synthesized oligonucleotides from the solid support was performed using AMA (1:1, 28% aqueous ammonia and 40% aqueous methylamine) at 65 C for 30 minutes or at room temperature for 5 hours for MMt-protected RNAs. The supernatant was then removed from the CPG via filtration and the solution was concentrated by speed-vac. The dried oligonucleotides was then dissolved in a mixture of DMSO/TEA 3HF/TEA (43:35:22) and headed to 65° C. for 2.5 hours with shaking. The deprotection reaction was quenched using 3M sodium acetate followed by isopropanol precipitation to yield a solid material. The oligos were then desalted using 3 kDa Amicon centrifugal filter, analyzed by LC-MS, and quantified using nanodrop. Oligos were then used without further purification or purified using IEX chromatography depending on purity. followed by ethanol precipitation to yield a solid material. At this stage oligos were purified using IEX chromatography based on purity. Exemplary nucleotides are listed below:

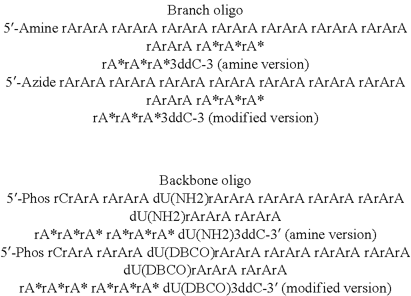

### 5′ Amine Deprotection of PSrA Oligonucleotides

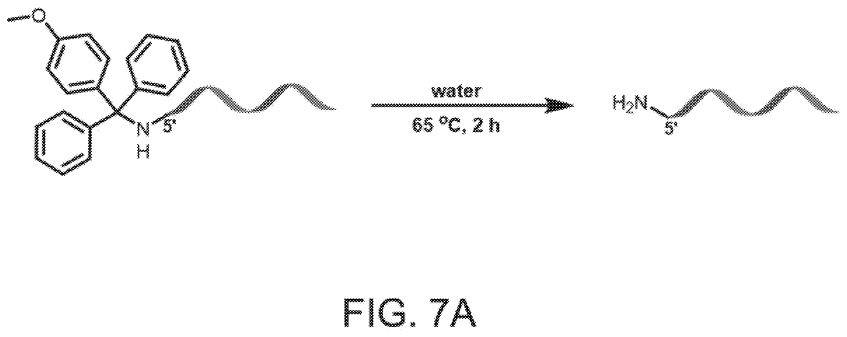

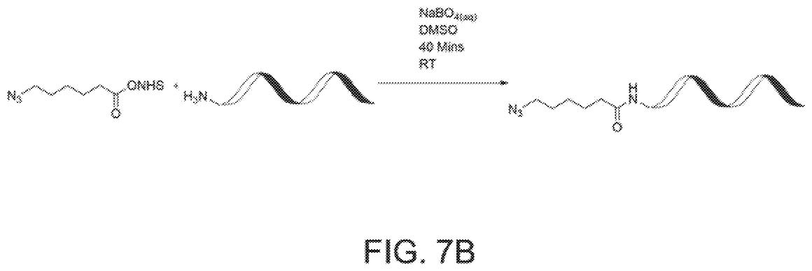

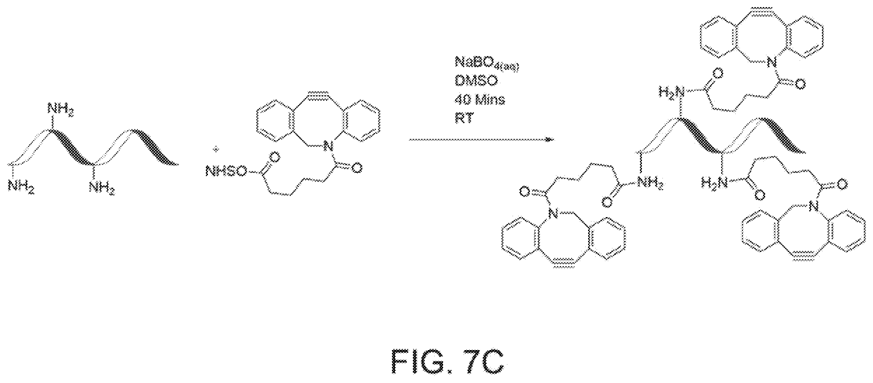

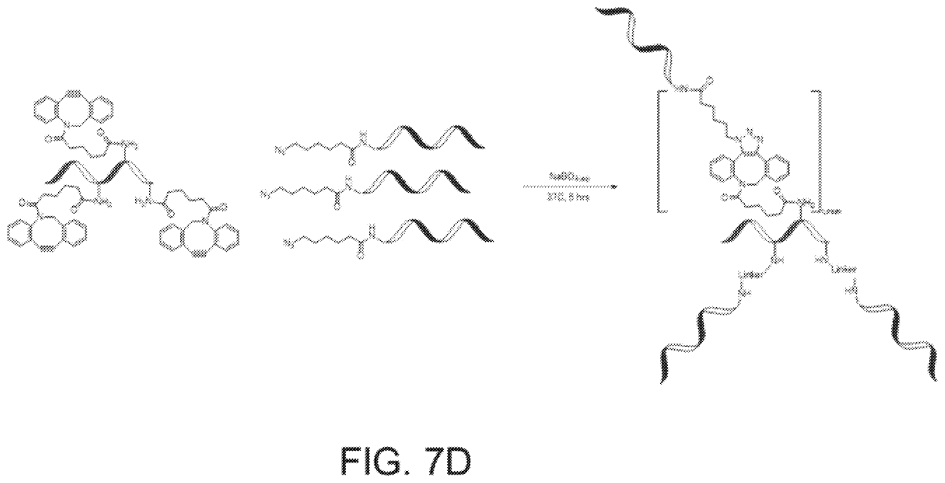

For RNAs containing 5′-MMt amino groups, the MMt was retained until the end of the RNA cleavage and deprotection to prevent any off-target amine reactivity. The MMt-protected RNA was dissolved in water (5 mg/mL) and heated to 65 C for 2 hours. The reaction is complete when the RNA solution becomes clear. Reaction workup was then performed by desalting using a 3 kDa Amicon centrifugal filter. FIG. 7A shows a reaction schematic of deprotection of 5′-amine oligonucleotides prior conjugation.

### Bioconjugation of 5′-Azide Via Amine/NHS Ester Strategy

A 50 mL falcon tube was charged with 5′-amine-modified oligonucleotides (5.6 μmol, 60 mg, 1 equiv.) from a stock solution, and concentrate the solution on a speedvac until dryness. Add 100 mM sodium borate buffer (6 mL, pH=8.4) and vortex until the 5′-amine-oligonucleotide was completely dissolved (if necessary, adjust pH of resultant solution to 8.4 using 500 mM sodium borate buffer (pH=9.5) or 1M TEAB buffer). Then, a solution of 5-Azidopentanoic acid N-hydroxysuccinimide ester (56 μmol, 13.4 mg, 10 equiv.) in DMSO (6 mL) was added to the reaction mixture and vortex. The reaction mixture was shaken on a thermomixer at 25° C., and 850 rpm for 40 min. After the completion of reaction, the product was isolated by ethanol precipitation. In short, 5 mL of sodium acetate (3M, pH=5.5) and 45 mL of absolute ethanol were added to the reaction. After vortexing, the mixture was kept at −80° C. for 1 hour, followed by centrifugation at 4000 rpm for 20 min. The pellet was recovered by removing the supernatant, washed the pellet with acetonitrile (2×10 mL) without breaking. The resultant pellet was dried on speedvac to obtain the desired 5′-azide-oligo with approx. 73% purity. The crude material was desalted and directly used for the further conjugation reactions. Desalting was done using a 3k Amicon spin column to obtain the desired pure compound in 58 mg, 95 isolated yield. FIG. 7B shows a reaction schematic of bioconjugation of 5′-Azide via Amine/NHS ester.

### Synthesis of Branched PSrA Tail Using Copper-Free DBCO/Azide Click Reaction

In a 5 mL eppendorf tube was aliquoted RNA (0.86 μmol, 10 mg,) containing three internal amine sites from stock a solution. Triethylammonium bicarbonate (pH=8.5) was added to give a final concentration of 100 mM. The solution was then concentrated by speed-vac. After, the RNA pellet was dissolved in 287 μL of 500 mM sodium borate buffer (pH=8.4). Separately, a solution of DBCO-NHS ester (10 μmol, 4.5 mg) was prepared in 574 μL DMSO. Once fully dissolved, the charged DMSO solution was added to the aqueous buffer containing RNA. The reaction was mixed by rapid pipetting and stirred at room temperature for 40 minutes. During mixing, the solution will often become turbid but clears quickly by rapid pipetting. After the reaction, 4 mL of 9:1 ethanol/sodium acetate (aq) (3 M, pH=5.0) was added and the solution was stored at −30 C for 1 hour to precipitate. The crude material was then desalted and directly used for the further conjugation reactions. Desalting was done using a 3k Amicon spin column to obtain the desired pure compound in 9.1 mg, 83% isolated yield. FIG. 7C shows a reaction schematic for conjugation of DBCO-NHS ester with internal amino RNA.

### Strain-Promoted Azide/Alkyne Cyclization (SPAAC) for Conjugation of Short RNA Fragments

In a 5 mL eppendorf tube was aliquoted RNA (3 mg, 234 nmols) containing three DBCO modified sites from a stock solution. Water and sodium borate buffer (500 mM, pH=8.4) were added to give a final buffer concentration of 100 mM and a final RNA concentration of 0.6 mM. Separately, azide-modified RNA (15 mg, 1,430 nmols) was aliquoted into 5 mL eppendorf tube and concentrated by speedvac. Once dried, the azide-modified RNA was dissolved in the borate buffer solution containing the DBCO-modified RNA. The solution was heated to 37 C with shaking for 6 hours. After the reaction, 9:1 ethanol/sodium acetate (aq) (3 M, pH=5.0) was added and the solution was stored at −30 C for 1 hour to precipitate. The crude material was then desalted using a 3k Amicon spin column to obtain the crude product. Quantification using standard RNA absorptivity, not sequence dependent absorptivity, indicated a recovery of 9.7 mg from the reaction. FIG. 7D shows conjugation of DBCO-modified oligo RNA with azide-modified RNA branches.

Post-Ligation Conjugation of DBCO-Modified chemRNA with Azide-Modified Branch

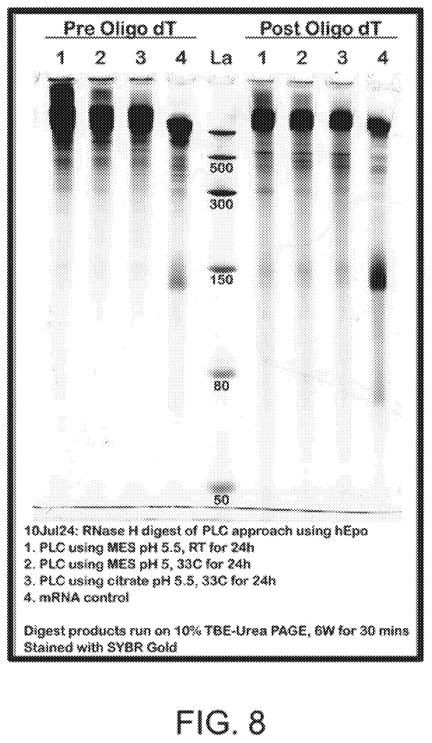

In 1.7 mL eppendorf tube was aliquoted Fluc mRNA (which had been ligated with the DBCO-modified oligo RNA by Imaly) (62 pmols, 39 μg). To the mRNA was added 500 mM MES buffer (pH=5.5), water, and azide-modified RNA (13 nmols, 13 μg) to give a final mRNA concentration of 2.5 UM and a MES concentration of 100 mM. Reaction was shaken at room temperature for 18 hours while protecting from light. After 18 hours, the reaction mixture was diluted with 400 μL of 50 mM Tris/100 mM KCl (pH=7.5) and the solution was desalted using a 100 kDa Amicon filter. To remove the azide, three washes with buffer were performed followed by two washes with deionized, nuclease-free water. Once concentrated, the branched chemRNA was quantified by nanodrop and analyzed by BioAnalyzer and gel-electrophoresis. Based on quantification, the yield for the reaction was 68% (26 μg). FIG. 7D shows Conjugation of DBCO-modified chemRNA with azide-modified RNA branches FIG. 8 shows Gel-based RNase H assay to assess tail integrity after post-ligation conjugation and unligated parent oligo would appear around the 150 Da band. Absence of this band indicates conjugation occurring on chemRNA.

mRNAs Having Branched polyA Tails Using Copper-Free Chemistries Improves mRNA Stability and Increased Protein Expression

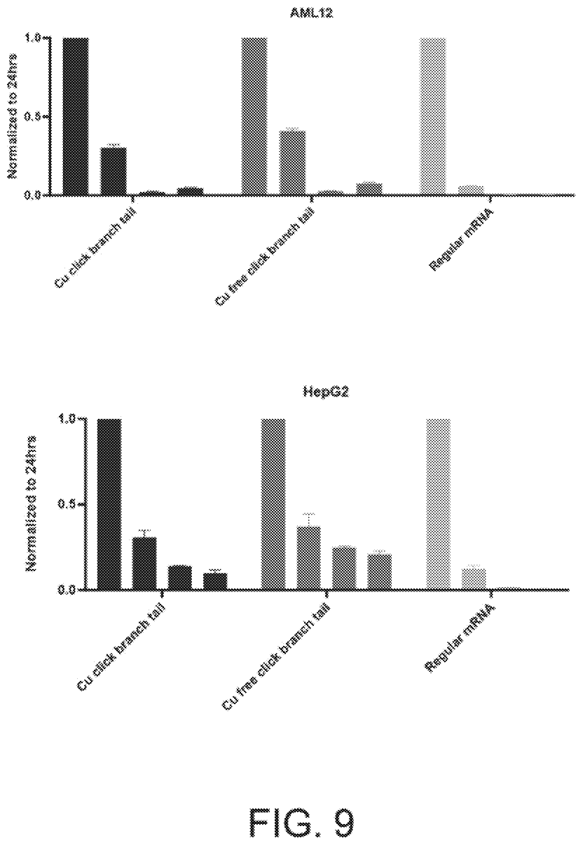

Translation stability of mRNA's having branched polyA tails using copper free click chemistry was evaluated using the cellular expression (AML12 cells and HepG2 cells) of the luciferase encoded by the mRNA. The luciferase activity was measured at day 1-4 after transfection with MessangerMax. The activity at each time point is normalized to day 1. As demonstrated in FIG. 9, the increase of the normalized value from day 2-4 indicates an increased stability of mRNAs having branched polyA tails using copper-free chemistries.

1. A multi-terminus polynucleotide comprising a first polyA polynucleotide linked to a second polyA polynucleotide, wherein the multi-terminus polyA polynucleotide comprises a formula represented by the structure: 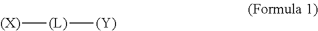
wherein:
(X) is a first functionalized nucleotide within the first polyA polynucleotide, wherein the functionalized nucleotide comprises an amino modification, an alkoxyamino modification, a thiol modification, a carboxyl modification, or an aldehyde modification;
(L) is a linker;
(Y) is a second functionalized nucleotide within the second polyA polynucleotide, wherein the functionalized nucleotide comprises an amino modification, an alkoxyamino modification, a carboxyl modification, or an aldehyde modification;
first functionalized nucleotide is linked to the linker through a linkage comprising an amide linkage, an oxime linkage, or a thioether linkage; and
the linker is linked to the second functionalized nucleotide is linked to the linker through a linkage comprising an amide linkage, an oxime linkage, or a thioether linkage.

2. The multi-terminus polynucleotide claim of 1, wherein (X) is located at a non-terminal position of the first polyA polynucleotide.

3. The multi-terminus polynucleotide of claim 2, wherein (Y) is located at a terminal or a non-terminal position of the second polyA polynucleotide.

4. The multi-terminus polynucleotide of claim 1, wherein the linker comprises a heteroalkyl linker.

5. The multi-terminus polynucleotide of claim 4, wherein the heteroalkyl linker comprises a polyethylene glycol (PEG) linker.

6. The multi-terminus polynucleotide of claim 1, wherein the first polyA polynucleotide and/or second polyA polynucleotide comprise a nucleobase sequence having 75% or greater adenine.

7. A modified ribonucleic acid molecule comprising, 5′ to 3, a 5′ cap, a region encoding a polypeptide, and a multi-terminus polyA polynucleotide comprising a first polyA polynucleotide linked to a second polyA polynucleotide, wherein the multi-terminus polyA polynucleotide comprises a formula represented by the structure: 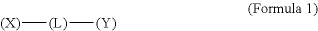
wherein:
(X) is a first functionalized nucleotide within the first polyA polynucleotide, wherein the functionalized nucleotide comprises an amino modification, an alkoxyamino modification, a thiol modification, a carboxyl modification, or an aldehyde modification;
(L) is a linker;
(Y) is a second functionalized nucleotide within the second polyA polynucleotide, wherein the functionalized nucleotide comprises an amino modification, an alkoxyamino modification, a carboxyl modification, or an aldehyde modification;
first functionalized nucleotide is linked to the linker through a linkage comprising an amide linkage, an oxime linkage, or a thioether linkage; and
the linker is linked to the second functionalized nucleotide is linked to the linker through a linkage comprising an amide linkage, an oxime linkage, or a thioether linkage.

8. The modified ribonucleic acid molecule of claim 7, wherein the linker is attached to (Y) through a second linkage comprising a second amide linkage or a thiol linkage.

9. The modified ribonucleic acid molecule of claim 7, wherein (X) is located at a non-terminal position of the first polyA polynucleotide.

10. The modified ribonucleic acid molecule of claim 7, wherein (Y) is located at a terminal or a non-terminal position of the second polyA polynucleotide.

11. The modified ribonucleic acid molecule of claim 7, wherein the linker comprises a heteroalkyl linker.

12. The modified ribonucleic acid molecule of claim 11, wherein the heteroalkyl linker comprises a polyethylene glycol (PEG) linker.

13. The modified ribonucleic acid molecule claim 7, wherein modified ribonucleic acid molecule further comprises a 5′ UTR located 3′ to the 5′ cap and 5′ to the region encoding the polypeptide.

14. The modified ribonucleic acid molecule of claim 13, wherein modified ribonucleic acid molecule further comprises a 3′ UTR located 3′ to the region encoding the polypeptide and 5′ to multi-terminus polyA polynucleotide.

15. A multi-terminus polynucleotide comprising a first polyA polynucleotide linked to a second polyA polynucleotide, wherein the multi-terminus polyA polynucleotide comprises a formula represented by the structure: 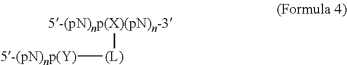
wherein:
(X) is a first functionalized nucleotide within the first polyA polynucleotide;
(L) is a heteroalkyl linker;
(Y) is a second functionalized nucleotide at the 3′ position of a second polyA polynucleotide;
each (p) is independently an inter-nucleoside linkage or terminal phosphate or phosphorothioate;
each (N) is independently a nucleotide or a modified nucleotide;
each (n) is independently an integer equal to or greater than 1;
the first functionalized nucleotide is linked to the linker; and
the linker is linked to the second functionalized nucleotide.

16. A method of increasing translation of a polypeptide, the method comprising:
contacting a cell with (i) a messenger ribonucleic acid molecule comprising the multi-terminus polynucleotide of acclaim 1.

17. The method of claim 16, wherein increasing translation comprises increasing the total amount of the polypeptide.

18. The method of any one claim 16, wherein increasing translation comprises increasing the duration of protein translation.

19. A method of increasing the half-life of a ribonucleic acid molecule, the method comprising:
attaching the multi-terminus polynucleotide of claim 1 to the 3′ end of the ribonucleic acid molecule.

20. The method of claim 19, wherein the ribonucleic acid molecule is a messenger ribonucleic acid molecule.

21. A composition comprising modified ribonucleic acid molecule comprising, 5′ to 3, a 5′ cap, a region encoding a polypeptide, and a multi-terminus polyA polynucleotide comprising a first polyA polynucleotide linked to a second polyA polynucleotide, wherein the multi-terminus polyA polynucleotide comprises a formula represented by the structure: 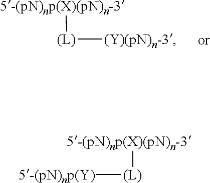
wherein:
(X) is a first functionalized nucleotide within the first polyA polynucleotide;
(L) is a heteroalkyl linker;
(Y) is a second functionalized nucleotide at the 3′ position of a second polyA polynucleotide;
each (p) is independently an inter-nucleoside linkage or terminal phosphate or phosphorothioate;
each (N) is independently a nucleotide or a modified nucleotide;
each (n) is independently an integer equal to or greater than 1;
the first functionalized nucleotide is linked to the linker;
the linker is linked to the second functionalized nucleotide; and
the composition is substantially free of copper.

22. The composition of claim 21 or claim 22, wherein the composition is substantially free of copper trace copper.
